(19) United States
(12) Patent Application Publication
(10) Pub. No.: US 2025/0000853 A1
(43) Pub. Date: Jan. 2, 2025

(54) COMBINATION OF A PD-1 ANTAGONIST AND A VEGF-R/FGFR/RET TYROSINE KINASE INHIBITOR FOR TREATING CANCER

(71) Applicants: EISAI R&D MANAGEMENT CO., LTD., Tokyo (JP); Merck Sharp & Dohme LLC, Rahway, NJ (US)

(72) Inventors: Andrew Evan Denker, North Wales, PA (US); Yu Kato, Tokyo (JP); Kimiyo Tabata, Tokyo (JP); Yusaku Hori, Tokyo (JP)

(21) Appl. No.: 18/826,658

(22) Filed: Sep. 6, 2024

Related U.S. Application Data
(63) Continuation of application No. 17/502,962, filed on Oct. 15, 2021, now Pat. No. 12,083,112.
Application No. 17/502,962 is a continuation of application No. 15/554,577, filed on Aug. 30, 2017, now Pat. No. 11,547,705.
(60) Provisional application No. 62/171,615, filed on Jun. 5, 2015.
(60) Provisional application No. 62/128,232, filed on Mar. 4, 2015.

(51) Int. Cl. A61K 31/47; A61K 39/00; A61K 39/395; A61K 45/06; C07K 16/28

(52) U.S. Cl. CPC A61K 31/47; A61K 39/39558; A61K 45/06; C07K 16/2818; C07K 16/2827; A61K 2039/505; A61K 2300/00; C07K 2317/76

(57) ABSTRACT

The present disclosure describes combination therapies comprising an antagonist of Programmed Death 1 receptor (PD-1) and a multi-RTK inhibitor, and the use of the combination therapies for the treatment of cancer. The multi-RTK inhibitor may be represented by Formula (I):

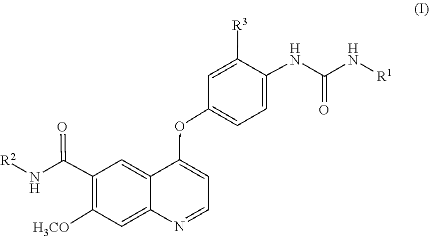

wherein R¹ is C₁₋₆ alkyl or C₃₋₈ cycloalkyl, R² is a hydrogen atom or C₁₋₆ alkoxy, and R³ is a hydrogen atom or a halogen atom. A tumor therapeutic agent is disclosed that combines a compound or pharmaceutically acceptable salt thereof represented by Formula I and an anti-PD-1 antibody.

### CROSS REFERENCE TO RELATED APPLICATIONS

This application is a continuation of Ser. No. 17/502,962, filed Oct. 15, 2021, allowed, which is a continuation of U.S. Application Ser. No. 15/554,577, filed on Aug. 30, 2017, pending, which is the National Stage of International Application No. PCT/US2016/020747, filed on Mar. 3, 2016, and which claims benefit to Japan Application JP 2015-042683 filed on Mar. 4, 2015, Japan Application JP 2015-114890 filed on Jun. 5, 2015, U.S. Provisional Application No. 62/128,232 filed Mar. 4, 2015, and U.S. Provisional Application 62/171,615 filed on Jun. 5, 2015, the contents of which are hereby incorporated by reference in their entirety.

### SEQUENCE LISTING

This application contains a sequence listing that has been submitted in XML format via EFS-Web and incorporated herein by reference in its entirety. Said XML copy, created on Aug. 16, 2024, is named 50000C_SeqListing.xml and is 34,675 bytes in size.

### TECHNICAL FIELD

Combination therapies useful for the treatment of cancer are disclosed. In particular, a combination therapy which comprises an antagonist of a Programmed Death 1 protein (PD-1) and a multiple receptor tyrosine kinase (multi-RTK) inhibitor is disclosed. Even more particularly, disclosed is a tumor therapeutic agent containing a combination of a quinoline derivative that exhibits a multi-tyrosine kinase inhibitory action and an anti-PD-1 antibody.

### BACKGROUND

PD-1 is recognized as an important player in immune regulation and the maintenance of peripheral tolerance. PD-1 is moderately expressed on naive T-, B- and Natural killer T (NKT)-cells and up-regulated by T/B-cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). It has been proposed that PD-L1 expressing tumor cells interact with PD-1 expressing T cells to attenuate T cell activation and evasion of immune surveillance, thereby contributing to an impaired immune response against the tumor. Therefore, an antibody directed against either the PD-1 receptor or the PD-L1 ligand can inhibit the binding therebetween, resulting in an increased immune action on the tumor cells (23).

Several monoclonal antibodies that inhibit the interaction between PD-1 and one or both of its ligands PD-L1 and PD-L2 are in clinical development for treating cancer. It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents.

Tyrosine kinases are involved in the modulation of growth factor signaling and thus are an important target for cancer therapies. Lenvatinib mesilate, discovered and developed by Eisai Co., Ltd., is an oral receptor tyrosine kinase (RTK) inhibitor that selectively inhibits the kinase activities of vascular endothelial growth factor (VEGF) receptors (VEGFR1 (FLT1), VEGFR2 (KDR) and VEGFR3 (FLT4)), and fibroblast growth factor (FGF) receptors FGFR1, 2, 3 and 4 in addition to other proangiogenic and oncogenic pathway-related RTKs (including the platelet-derived growth factor (PDGF) receptor PDGFRα; KIT; and the RET proto-oncogene (RET)) involved in tumor proliferation. In particular, lenvatinib possesses a new binding mode (Type V) to VEGFR2, as confirmed through X-ray crystal structural analysis, and exhibits rapid and potent inhibition of kinase activity, according to kinetic analysis.

Lenvatinib mesilate was recently approved in the United States for the treatment of patients with locally recurrent or metastatic, progressive, radioactive iodine-refractory differentiated thyroid cancer. Its chemical name is 4-[3-chloro-4-(cyclopropylaminocarbonyl) aminophenoxy]-7-methoxy-6-quinolinecarboxamide methanesulfonate. Eisai was granted Orphan Drug Designation (ODD) for lenvatinib mesilate in various types of thyroid cancer in the United States, Japan, and Europe. Lenvatinib mesilate is under investigation in thyroid, hepatocellular, endometrial, non-small cell lung cancer, renal cell carcinoma (RCC), melanoma, glioblastoma, and other solid tumor types. The compound represented by Formula (I) below has anti-angiogenic actions (17), inhibitory effects (18-21) against tyrosine kinases which are reported to be involved in malignant alteration of tumors and the like.

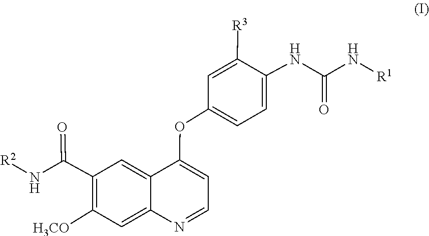

R¹ is C₁₋₆ alkyl or C₃₋₈ cycloalkyl. R² is a hydrogen atom or C₁₋₆ alkoxy. R³ is a hydrogen atom or a halogen atom.

In general, tumor therapeutic agents are often not effective for all of the patients when administered individually. Thus, attempts have been made to increase the cure rate of such therapeutic agents by combining them (22).

### SUMMARY

As disclosed herein, administration of a combination of a compound represented by Formula (I) and a PD-1 receptor antibody attains an unexpectedly excellent anti-tumor effect.

A method is provided for treating a cancer in an individual that includes administering to the individual a combination therapy which comprises a PD-1 antagonist, which is not MPDL3280A, and a multi-RTK inhibitor. In some instances, the individual is a human. The cancer may be a solid tumor, a non-small cell lung cancer (NSCLC), RCC, endometrial cancer, urothelial cancer, squamous cell carcinoma of head and neck or melanoma.

The PD-1 antagonist of the method may be a monoclonal antibody or an antigen binding fragment thereof. In some instances, the antagonist is an anti-PD-1 antibody. The antagonist may be pembrolizumab or nivolumab.

The multi-RTK inhibitor of the method may be a compound or pharmaceutically acceptable salt thereof represented by Formula (I):

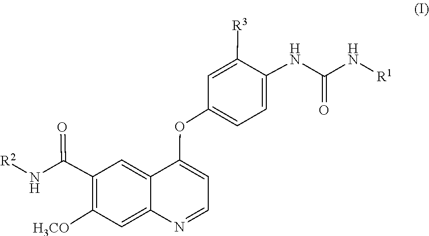

in which R¹ is C₁₋₆ alkyl or C₃₋₈ cycloalkyl, R² is a hydrogen atom or C₁₋₆ alkoxy, and R³ is a hydrogen atom or a halogen atom. The compound represented by Formula (I) may be one or more of the following compounds:
4-[3-chloro-4-(cyclopropylaminocarbonyl)aminophenoxy]-7-methoxy-6-quinolinecarboxamide:

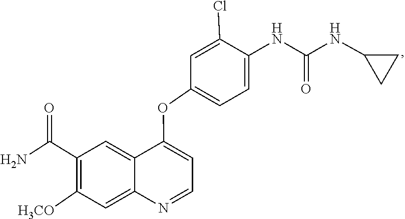 [SMILES: COc1cc2nccc(Oc3ccc(NC(=O)NC4CC4)c(Cl)c3)c2cc1C(N)=O]

4-[3-chloro-4-(methylaminocarbonyl)aminophenoxy]-7-methoxy-6-quinolinecarboxamide:

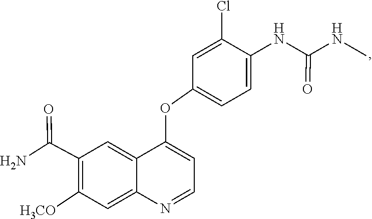 [SMILES: CNC(=O)Nc1ccc(Oc2ccnc3cc(OC)c(C(N)=O)cc23)cc1Cl]

4-[3-chloro-4-(ethylaminocarbonyl)aminophenoxy]-7-methoxy-6-quinolinecarboxamide:

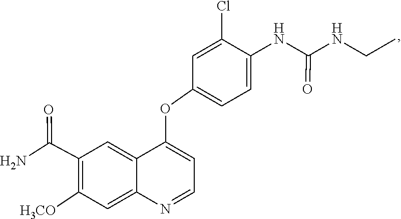 [SMILES: CCNC(=O)Nc1ccc(Oc2ccnc3cc(OC)c(C(N)=O)cc23)cc1Cl]

N6-methoxy-4-(3-chloro-4-{[(cyclopropylamino)carbonyl)amino]phenoxy}-7-methoxy-6-quinolinecarboxamide:

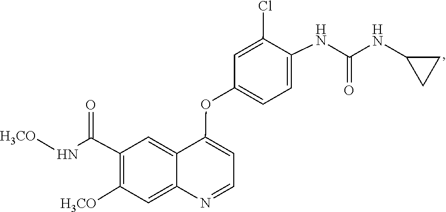 [SMILES: CONC(=O)c1cc2c(Oc3ccc(NC(=O)NC4CC4)c(Cl)c3)ccnc2cc1OC]

and N6-methoxy-4-(3-chloro-4-{[(ethylamino)carbonyl]amino}phenoxy)-7-methoxy-6-quinolinecarboxamide:

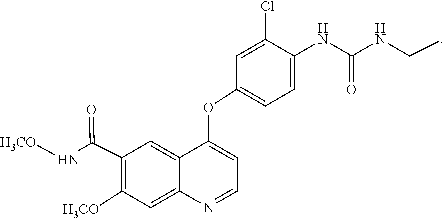 [SMILES: CCNC(=O)Nc1ccc(Oc2ccnc3cc(OC)c(C(=O)NOC)cc23)cc1Cl]

More specifically, the compound represented by Formula (I) is 4-[3-chloro-4-(cyclopropylaminocarbonyl)aminophenoxy]-7-methoxy-6-quinolinecarboxamide:

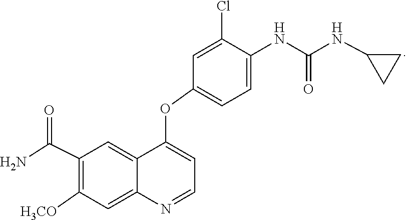 [SMILES: COc1cc2nccc(Oc3ccc(NC(=O)NC4CC4)c(Cl)c3)c2cc1C(N)=O]

In some instances, the PD-1 antagonist of the method is pembrolizumab and the multi-RTK inhibitor is lenvatinib or a pharmaceutically acceptable salt thereof. Administration of pembrolizumab may occur after the administration of lenvatinib in some treatment regimens. In some instances, the lenvatinib is administered after the pembrolizumab.

A kit is provided that includes a first container, a second container and a package insert. The first container of the kit includes at least one dose of a medicament comprising a PD-1 antagonist, which is not MPDL3280A, and the second container includes at least one dose of a medicament comprising a multi-RTK inhibitor. The package insert includes instructions for treating an individual for cancer using the medicaments. The instructions of the kit may state that the medicaments are intended for use in treating an individual having a cancer that tests positive for PD-L1 expression by an immunohistochemical (IHC) assay. In some instances, the individual may be a human.

The multi-RTK inhibitor of the kit may be is a compound or pharmaceutically acceptable salt thereof represented by Formula (I):

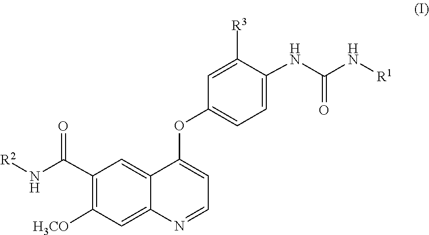

in which R¹ is C₁₋₆ alkyl or C₃₋₈ cycloalkyl, R² is a hydrogen atom or C₁₋₆ alkoxy, and R³ is a hydrogen atom or a halogen atom. The compound represented by Formula (I) may be one or more of the following compounds:
4-[3-chloro-4-(cyclopropylaminocarbonyl)aminophenoxy]-7-methoxy-6-quinolinecarboxamide:

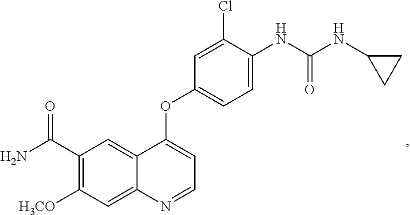 [SMILES: COc1cc2nccc(Oc3ccc(NC(=O)NC4CC4)c(Cl)c3)c2cc1C(N)=O]

4-[3-chloro-4-(methylaminocarbonyl)aminophenoxy]-7-methoxy-6-quinolinecarboxamide:

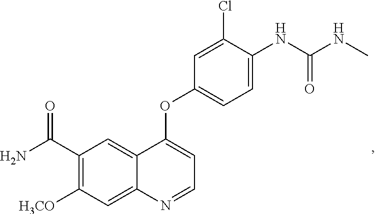 [SMILES: CNC(=O)Nc1ccc(Oc2ccnc3cc(OC)c(C(N)=O)cc23)cc1Cl]

4-[3-chloro-4-(ethylaminocarbonyl)aminophenoxy]-7-methoxy-6-quinolinecarboxamide:

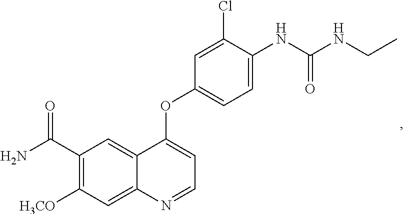 [SMILES: CCNC(=O)Nc1ccc(Oc2ccnc3cc(OC)c(C(N)=O)cc23)cc1Cl]

N6-methoxy-4-(3-chloro-4-{[(cyclopropylamino)carbonyl)amino]phenoxy}-7-methoxy-6-quinolinecarboxamide:

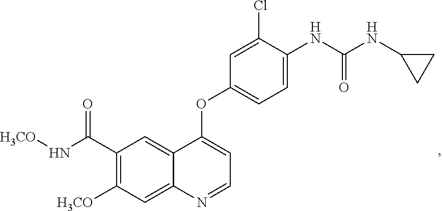 [SMILES: CONC(=O)c1cc2c(Oc3ccc(NC(=O)NC4CC4)c(Cl)c3)ccnc2cc1OC]

and N6-methoxy-4-(3-chloro-4-{[(ethylamino)carbonyl]amino}phenoxy)-7-methoxy-6-quinolinecarboxamide:

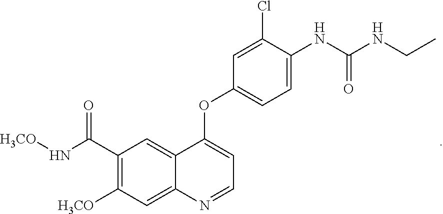 [SMILES: CCNC(=O)Nc1ccc(Oc2ccnc3cc(OC)c(C(=O)NOC)cc23)cc1Cl]

More specifically, the compound represented by Formula (I) may be 4-[3-chloro-4-(cyclopropylaminocarbonyl)aminophenoxy]-7-methoxy-6-quinolinecarboxamide:

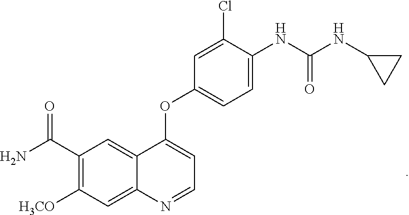 [SMILES: COc1cc2nccc(Oc3ccc(NC(=O)NC4CC4)c(Cl)c3)c2cc1C(N)=O]

The PD-1 antagonist provided by the kit may be pembrolizumab formulated as a liquid medicament which comprises 25 mg/ml pembrolizumab, 7% (w/v) sucrose, 0.02% (w/v) polysorbate 80 in 10 mM histidine buffer pH 5.5, and the multi-RTK inhibitor may be lenvatinib or a pharmaceutically acceptable salt thereof formulated as a 4 mg or 10 mg lenvatinib capsule comprising calcium carbonate, mannitol, microcrystalline cellulose, hydroxypropylcellulose, low-substituted hydroxypropylcellulose, and talc.

The medicaments included with the kit may be applied to treat NSCLC, RCC, endometrial cancer, urothelial cancer, squamous cell carcinoma of head and neck or melanoma.

A method is provided for treating a human individual diagnosed with a cancer, comprising administering to the individual a combination therapy for at least 24 weeks. The combination therapy includes pembrolizumab and lenvatinib or a pharmaceutically acceptable salt thereof. Lenvatinib or a pharmaceutically acceptable salt thereof may be administered at a daily dose of 24 mg, 20 mg or 14 mg, each as lenvatinib, and pembrolizumab may be administered at a dose of 200 mg Q3W.

A medicament is provided comprising a PD-1 antagonist for use in combination with a multi-RTK inhibitor for treating a cancer.

A medicament is also provided comprising a multi-RTK inhibitor for use in combination with a PD-1 antagonist for treating a cancer.

A use is also provided of a PD-1 antagonist in the manufacture of medicament for treating a cancer in an individual when administered in combination with a multi-RTK inhibitor and use of a multi-RTK inhibitor in the manufacture of a medicament for treating a cancer in an individual when administered in combination with a PD-1 antagonist.

Also provided is an use of a PD-1 antagonist and a multi-RTK inhibitor in the manufacture of medicaments for treating a cancer in an individual. Said medicaments can comprise a kit, and the kit can also comprises a package insert comprising instructions for using the PD-1 antagonist in combination with a multi-RTK inhibitor to treat a cancer in an individual. The kit may include a pharmaceutical composition comprising a compound or pharmaceutically acceptable salt thereof represented by the above Formula (I) and a vehicle. The kit may include a pharmaceutical composition comprising an anti-PD-1 antibody and a vehicle.

In all of the above treatment methods, medicaments and uses, the PD-1 antagonist inhibits the binding of PD-L1 to PD-1, and preferably also inhibits the binding of PD-L2 to PD-1. In some of the above treatment methods, medicaments and uses, the PD-1 antagonist is a monoclonal antibody, or an antigen binding fragment thereof, which specifically binds to PD-1 or to PD-L1 and blocks the binding of PD-L1 to PD-1. For example, the PD-1 antagonist can be an anti-PD-1 antibody which comprises a heavy chain and a light chain, and wherein the heavy and light chains comprise the amino acid sequences shown in FIG. 6 (SEQ ID NO:21 and SEQ ID NO: 22). The anti-PD-1 antibody may be combined with a compound or pharmaceutically acceptable salt thereof represented by the above Formula (I) for therapy of a tumor. The anti-PD-1 antibody may be combined with a compound or pharmaceutically acceptable salt thereof represented by the above Formula (I). Also provided is a method of treating a tumor is disclosed that can include the combined use of a compound or pharmaceutically acceptable salt thereof represented by the above Formula (I) and an anti-PD-1 antibody.

In all of the above treatment methods, medicaments and uses herein, the multi-RTK inhibitor inhibits the kinase activities of at least each of the following RTKs: (i) VEGFR2, (ii) at least one FGFR selected from the group consisting of FGFR1, 2, 3 and 4; and (iii) RET. In some instances, the multi-RTK inhibitor also inhibits the kinase activities of VEGFR1, VEGFR3, fibroblast growth factor (FGF) receptors FGFR1, 2, 3 and 4, platelet-derived growth factor (PDGF) receptor alpha (PDGFRα); and KIT.

In some of the above treatment methods, medicaments and uses, the multi-RTK inhibitor is lenvatinib or a pharmaceutically acceptable salt thereof, such as lenvatinib mesilate.

In the above treatment methods, medicaments and uses, the individual is a human and the cancer is a solid tumor and in some instances, the solid tumor is bladder cancer, breast cancer, clear cell kidney cancer, squamous cell carcinoma of head and neck, lung squamous cell carcinoma, malignant melanoma, NSCLC, ovarian cancer, pancreatic cancer, prostate cancer, RCC, small-cell lung cancer (SCLC) or triple negative breast cancer. The cancer may be NSCLC, endometrial cancer, urothelial cancer, squamous cell carcinoma of head and neck or melanoma.

In the above treatment methods, medicaments and uses, the individual can be a human and the cancer is a heme malignancy and in some instances, the heme malignancy is acute lymphoblastic leukemia (ALL), acute myeloid leukemia (AML), chronic lymphocytic leukemia (CLL), chronic myeloid leukemia (CML), diffuse large B-cell lymphoma (DLBCL), EBV-positive DLBCL, primary mediastinal large B-cell lymphoma, T-cell/histiocyte-rich large B-cell lymphoma, follicular lymphoma, Hodgkin's lymphoma (HL), mantle cell lymphoma (MCL), multiple myeloma (M M), myeloid cell leukemia-1 protein (Mcl-1), myelodysplastic syndrome (MDS), non-Hodgkin's lymphoma (NHL), or small lymphocytic lymphoma (SLL).

Also, any of the above treatment methods, medicaments and uses can be utilized if the cancer tests positive for the expression of one or both of PD-L1 and PD-L2. In still other instances, the cancer has elevated PD-L1 expression.

In some of the above treatment methods, medicaments and uses, the individual is a human, the cancer tests positive for human PD-L1 and is selected from the group consisting of NSCLC, endometrial cancer, urothelial cancer, squamous cell carcinoma of head and neck, or melanoma.

In some of the above treatment methods, medicaments and uses, the multi-RTK inhibitor is a compound or pharmaceutically acceptable salt thereof represented by Formula (I):

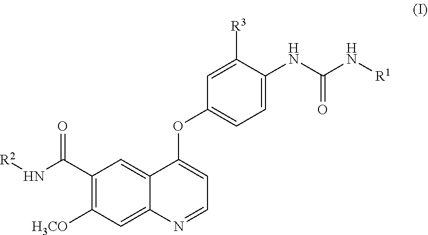

wherein R¹ is C₁₋₆ alkyl or C₃₋₈ cycloalkyl, R² is a hydrogen atom or C₁₋₆ alkoxy, and R³ is a hydrogen atom or a halogen atom. The tumor therapeutic agent may administer simultaneously or separately a compound or pharmaceutically acceptable salt thereof represented by the above Formula (I) and an anti-PD-1 antibody. The tumor therapeutic agent may be administered simultaneously or separately. For example, a composition can comprise a compound or pharmaceutically acceptable salt thereof represented by the above Formula (I), and a composition comprising an anti-PD-1 antibody. The tumor therapeutic agent may include a compound or pharmaceutically acceptable salt thereof represented by the above Formula (I), and an anti-PD-1 antibody.

A compound or pharmaceutically acceptable salt thereof is disclosed that is represented by the Formula (I) for combined use with an anti-PD-1 antibody. The compound or pharmaceutically acceptable salt thereof represented by the Formula (I) may be used for therapy of a tumor by combined use with an anti-PD-1 antibody.

A pharmaceutical composition is disclosed that may include a compound or pharmaceutically acceptable salt thereof represented by the above Formula (I), an anti-PD-1 antibody, and a vehicle.

The compound represented by the above Formula (I) is preferably one or more of the following compounds:
4-[3-chloro-4-(cyclopropylaminocarbonyl)aminophenoxy]-7-methoxy-6-quinolinecarboxamide:

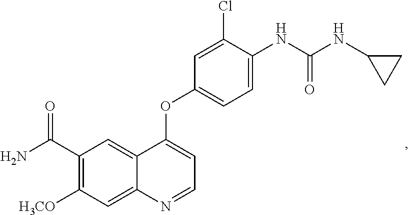 [SMILES: COc1cc2nccc(Oc3ccc(NC(=O)NC4CC4)c(Cl)c3)c2cc1C(N)=O]

4-[3-chloro-4-(methylaminocarbonyl)aminophenoxy]-7-methoxy-6-quinolinecarboxamide:

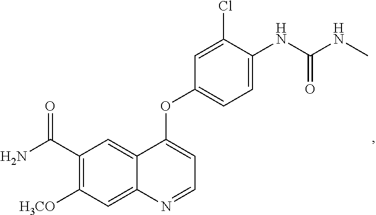 [SMILES: CNC(=O)Nc1ccc(Oc2ccnc3cc(OC)c(C(N)=O)cc23)cc1Cl]

4-[3-chloro-4-(ethylaminocarbonyl)aminophenoxy]-7-methoxy-6-quinolinecarboxamide:

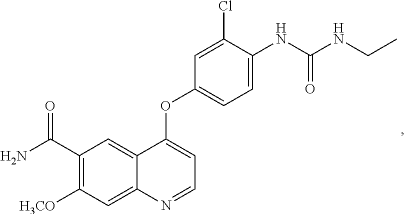 [SMILES: CCNC(=O)Nc1ccc(Oc2ccnc3cc(OC)c(C(N)=O)cc23)cc1Cl]

N6-methoxy-4-(3-chloro-4-{[(cyclopropylamino)carbonyl)amino]phenoxy}-7-methoxy-6-quinolinecarboxamide:

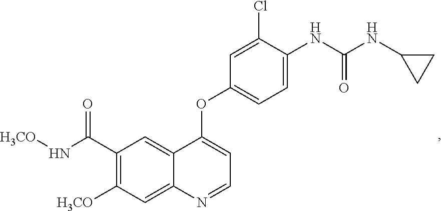 [SMILES: CONC(=O)c1cc2c(Oc3ccc(NC(=O)NC4CC4)c(Cl)c3)ccnc2cc1OC]

And N6-methoxy-4-(3-chloro-4-{[(ethylamino)carbonyl]amino}phenoxy)-7-methoxy-6-quinolinecarboxamide:

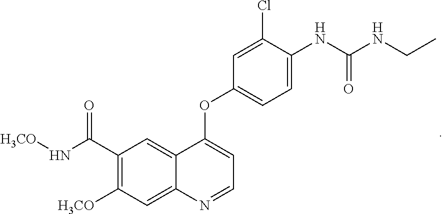 [SMILES: CCNC(=O)Nc1ccc(Oc2ccnc3cc(OC)c(C(=O)NOC)cc23)cc1Cl]

The compound represented by the above Formula (I) is more preferably 4-[3-chloro-4-(cyclopropylaminocarbonyl)aminophenoxy]-7-methoxy-6-quinolinecarboxamide:

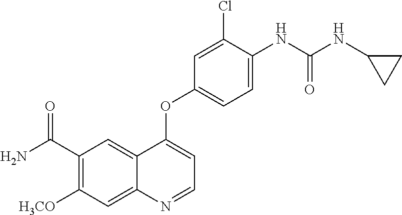 [SMILES: COc1cc2nccc(Oc3ccc(NC(=O)NC4CC4)c(Cl)c3)c2cc1C(N)=O]

(hereinafter, also sometimes indicated as lenvatinib).

A tumor therapeutic agent is provided for combined use of a compound having a multi-tyrosine kinase inhibitory action and an anti-PD-1 antibody. Such a tumor therapeutic agent exhibits an excellent anti-tumor effect compared to cases where these are individually used, and may exhibit anti-tumor effects against various cancer types.

### BRIEF DESCRIPTION OF THE DRAWINGS

FIG. 1 shows amino acid sequences of the light chain and heavy chain CDRs for an exemplary anti-PD-1 monoclonal antibody (SEQ ID NOs: 1-6).

FIG. 2 shows amino acid sequence of the light chain and heavy chain CDRs for another exemplary anti-PD-1 monoclonal antibody (SEQ ID NOs: 7-12).

FIGS. 3A and 3B shows amino acid sequences of the heavy chain variable region (FIG. 3A) and full length heavy chain (FIG. 3B) for an exemplary anti-PD-1 monoclonal antibody (SEQ ID NO:13 and SEQ ID NO:14).

FIG. 4 shows amino acid sequences of alternative light chain variable regions for an exemplary anti-PD-1 monoclonal antibody (SEQ ID NOs: 15-17).

FIGS. 5A and 5B show amino acid sequences of alternative light chains for an exemplary anti-PD-1 monoclonal antibody, with FIG. 5A showing the amino acid sequences for the K09A-L-11 and K09A-L-16 light chains (SEQ ID NOs: 18 and 19, respectively) and FIG. 5B showing the amino acid sequence for the K09A-L-17 light chain (SEQ ID NO:20).

FIG. 6 shows amino acid sequences of the heavy and light chains for pembrolizumab (SEQ ID NOs. 21 and 22, respectively).

FIG. 7 shows amino acid sequences of the heavy and light chains for nivolumab (SEQ ID NOS: 23 and 24, respectively).

FIG. 8 is a diagram illustrating anti-tumor effects of lenvatinib, an anti-PD-1 antibody (RMP1-14), and a combination of both in a subcutaneous LL/2 (LLc1) transplantation model.

FIG. 9 shows the anti-cancer or -tumor effect on the eleventh day since commencing treatment in a mouse model with colon cancer as disclosed herein.

FIG. 10 is a graph of the tumor volume plotted by days subsequent to administration for the control group, lenvatinib or PD-L1 individually, and a combination of lenvatinib and PD-L1 as disclosed herein.

### DETAILED DESCRIPTION

Abbreviations. Throughout the detailed description and examples the following abbreviations will be used:
BOR Best overall response
BID One dose twice daily
CBR Clinical Benefit Rate
CDR Complementarity determining region
CHO Chinese hamster ovary
CR Complete Response
DCR Disease Control Rate
DFS Disease free survival
DLT Dose limiting toxicity
DOR Duration of Response
DSDR Durable Stable Disease Rate
FFPE Formalin-fixed, paraffin-embedded
FR Framework region
IgG Immunoglobulin G
IHC Immunohistochemistry or immunohistochemical
irRC Immune related response criteria
IV Intravenous
MTD Maximum tolerated dose
NCBI National Center for Biotechnology Information
NCI National Cancer Institute
ORR Objective response rate
OS Overall survival
PD Progressive disease
PD-1 Programmed Death 1
PD-L1 Programmed Cell Death 1 Ligand 1
PD-L2 Programmed Cell Death 1 Ligand 2
PFS Progression free survival
PR Partial response
Q2W One dose every two weeks
Q3W One dose every three weeks
QD One dose per day
RECIST Response Evaluation Criteria in Solid Tumors
SD
Stable disease
Immunoglobulin heavy chain variable region
VH
VK Immunoglobulin kappa light chain variable region

### I. DEFINITIONS

So that the methods, compositions, and uses may be more readily understood, certain technical and scientific terms are specifically defined below. Unless specifically defined elsewhere in this document, all other technical and scientific terms used herein have the meaning commonly understood by one of ordinary skill in the art.

“About” when used to modify a numerically defined parameter (e.g., the dose of a PD-1 antagonist or a multi-RTK inhibitor, or the length of treatment time with a combination therapy described herein) means that the parameter may vary by as much as 10% below or above the stated numerical value for that parameter. For example, a dose of about 20 mg may vary between 18 mg and 22 mg.

“Preferably” means a more desirable choice. For example, when used to modify a numerically defined parameter it indicates that the preferred parameter provides an improved result over another value for the parameter. This meaning of “preferably” only applies outside of the United States.

As used herein, including the appended claims, the singular forms of words such as “a,” “an,” and “the,” include their corresponding plural references unless the context clearly dictates otherwise.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies, and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, FUNDAMENTAL IMMUNOLOGY Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of SEQUENCES OF PROTEINS OF IMMUNOLOGICAL INTEREST, Kabat, et al.; National Institutes of Health, Bethesda, Md.; 5^{th} ed.; NIH Publ. No. 91-3242 (1991); Kabat (1978) Adv. Prot. Chem. 32:1-75; K abat, et al., (1977) J. Biol. Chem. 252: 6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) SEQUENCES OF PROTEINS OF IMMUNOLOGICAL INTEREST, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50: 1S-10S (2009); Eisenhauer et al., supra). In some instances, an anti-tumor response to a combination therapy described herein is assessed using RECIST 1.1 criteria (response evaluation criteria in solid tumors), bidimentional irRC (immune related response criteria), or unidimensional irRC. In some instances, an anti-tumor response is any of SD, PR, CR, PFS, or DFS.

“Bidimensional irRC” refers to the set of criteria described in Wolchok J D, et al. “Guidelines for the evaluation of immune therapy activity in solid tumors: immune-related response criteria,” Clin Cancer Res. 2009; 15 (23): 7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response. Classes of biotherapeutic agents include, but are not limited to, antibodies to VEGF, epidermal growth factor receptor (EGFR), Her2/neu, other growth factor receptors, CD20, CD40, CD-40L, CTLA-4, OX-40, 4-1BB, and ICOS.

The terms “cancer,” “cancerous,” “tumor,” or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, adenoma, neurilemmoma, gastrointestinal (tract) cancer, gastric cancer, renal cancer, gallbladder cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, endometrial cancer, uterine body cancer, uterine cervical cancer, and head and neck cancer. Another particular example of cancer includes renal cell carcinoma. A further particular example of cancer includes clear cell kidney cancer. Cancers that may be treated in accordance with the disclosed treatment methods, medicaments, and disclosed uses include those characterized by elevated expression of one or both of PD-L1 and PD-L2 in tested tissue samples.

“CBR” or “Clinical Benefit Rate” means CR+PR+durable SD.

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods disclosed herein include cytostatic and/or cytotoxic agents.

“Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the disclosed treatment methods, medicaments, and disclosed uses, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“DCR” or “Disease Control Rate” means CR+PR+SD.

“Diagnostic anti-PD-L monoclonal antibody” means a mAb which specifically binds to the mature form of the designated PD-L (PD-L1 or PDL2) that is expressed on the surface of certain mammalian cells. A mature PD-L lacks the presecretory leader sequence, also referred to as leader peptide The terms “PD-L” and “mature PD-L” are used interchangeably herein, and will be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 25)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

Specific examples of diagnostic anti-human PD-L1 mAbs useful as diagnostic mAbs for IHC detection of PD-L1 expression in FFPE tumor tissue sections are antibody 20C3 and antibody 22C3, which are described in the copending international patent application PCT/US13/075932, filed 18 Dec. 2013 and published as WO2014/100079 on 26 Jun. 2014. Another anti-human PD-L1 mAb that has been reported to be useful for IHC detection of PD-L1 expression in FFPE tissue sections (Chen, B. J. et al., Clin Cancer Res 19:3462-3473 (2013)) is a rabbit anti-human PD-L1 mAb publicly available from Sino Biological, Inc. (Beijing, P. R. China; Catalog number 10084-R015).

“DSDR” or “Durable Stable Disease Rate” means SD for ≥23 weeks.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a Basic Local Alignment Search Tool (BLAST®) algorithm, which is a registered mark of the National Library of Medicine, wherein the parameters of the algorithm are selected to give the largest match between the respective sequences over the entire length of the respective reference sequences.

The following representative references relate to BLAST® algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” IN ATLAS OF PROTEIN SEQUENCE AND STRUCTURE, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” IN ATLAS OF PROTEIN SEQUENCE AND STRUCTURE, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” IN THEORETICAL AND COMPUTATIONAL METHODS IN GENOME RESEARCH (S. Suhai, ed.), (1997) pp. 1-14, Plenum, New York.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) SEQUENCES OF PROTEINS OF IMMUNOLOGICAL INTEREST, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the treatment methods, medicaments, and disclosed uses may be made by the hybridoma method first described by Kohler et al. (1975) Nature 256:495, or may be made by recombinant deoxyribonucleic acid (DNA) methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“Non-responsder patient”, when referring to a specific anti-tumor response to treatment with a combination therapy described herein, means the patient did not exhibit the anti-tumor response.

“ORR” or “objective response rate” refers in some instances to CR+PR, and ORR (week 24) refers to CR and PR measured using irRECIST in each patient in a cohort after 24 weeks of treatment with lenvatinib mesilate in combination with pembrolizumab.

“Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and disclosed uses in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. The PD-1 antagonist is not anti-PD-L1 monoclonal antibody MPDL3280A.

PD-1 antagonists useful in the any of the treatment methods, medicaments and disclosed uses include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. The human constant region is selected from the group consisting of IgG1, IgG2, IgG3 and IgG4 constant regions, and preferably the human constant region is an IgG1 or IgG4 constant region. In some instances, the antigen binding fragment is selected from the group consisting of Fab, Fab′-SH, F(ab′)₂, scFv and Fv fragments.

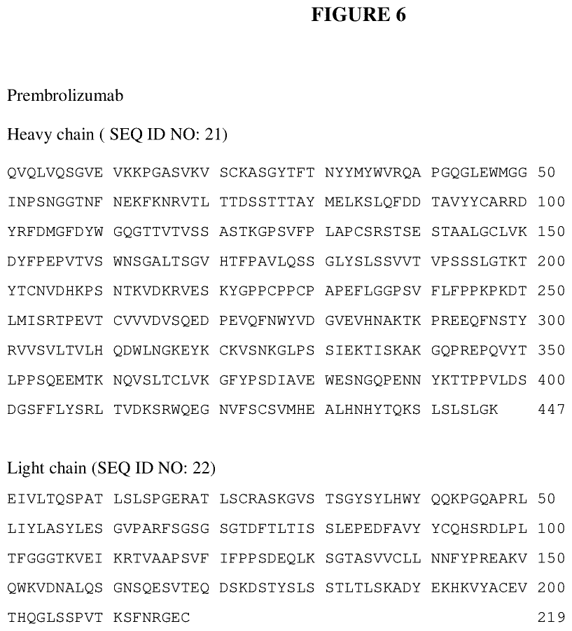

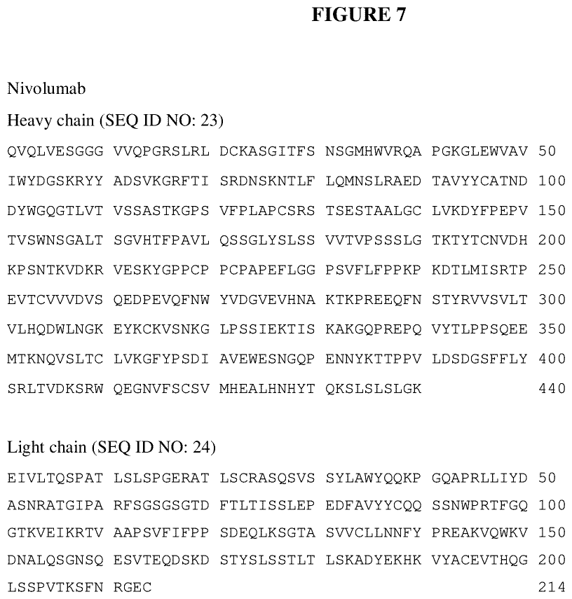

Examples of mAbs that bind to human PD-1, and useful in the treatment methods, medicaments and disclosed uses, are described in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist in the treatment methods, medicaments and disclosed uses include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in FIG. 6, nivolumab (BMS-936558), a human IgG4 mAb with the structure described in WHO Drug Information, Vol. 27, No. 1, pages 68-69 (2013) and which comprises the heavy and light chain amino acid sequences shown in FIG. 7, pidilizumab, a humanized monoclonal antibody, AMP-224, and AMP-514; the humanized antibodies h409A11, h409A16 and h409A17, which are described in WO2008/156712, and AMP-514, which is being developed by MedImmune.

Examples of mAbs that bind to human PD-L1, and useful in the treatment methods, medicaments and disclosed uses, are described in WO2013/019906, WO2010/077634 A1 and U.S. Pat. No. 8,383,796. Specific anti-human PD-L1 mAbs useful as the PD-1 antagonist in the treatment methods, medicaments and disclosed uses include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in the any of the treatment methods, medicaments and disclosed uses include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as a PD-1 antagonist in the treatment methods, medicaments and uses described herein include AMP-224 (also known as B7-DCIg), which is a PD-L2-FC fusion protein and binds to human PD-1.

The treatment methods, medicaments and disclosed uses provide for the PD-1 antagonist to be a monoclonal antibody, or antigen binding fragment thereof, which comprises: (a) light chain CDRs SEQ ID NOs: 1, 2 and 3 and heavy chain CDRs SEQ ID NOs: 4, 5 and 6; or (b) light chain CDRs SEQ ID NOs: 7, 8 and 9 and heavy chain CDRs SEQ ID NOs: 10, 11 and 12.

The treatment methods, medicaments and disclosed uses provide for the PD-1 antagonist to be a monoclonal antibody, or antigen binding fragment thereof, which specifically binds to human PD-1 and comprises (a) a heavy chain variable region comprising SEQ ID NO: 13 or a variant thereof, and (b) a light chain variable region comprising an amino acid sequence selected from the group consisting of SEQ ID NO: 15 or a variant thereof; SEQ ID NO: 16 or a variant thereof; and SEQ ID NO: 17 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

The PD-1 antagonist for any of the treatment methods, medicaments and disclosed uses, can be a monoclonal antibody which specifically binds to human PD-1 and comprises (a) a heavy chain comprising SEQ ID NO: 14 and (b) a light chain comprising SEQ ID NO: 18, SEQ ID NO:19 or SEQ ID NO:20.

The treatment methods, medicaments and disclosed uses provide for the PD-1 antagonist to be a monoclonal antibody which specifically binds to human PD-1 and comprises (a) a heavy chain comprising SEQ ID NO: 14 and (b) a light chain comprising SEQ ID NO:18.

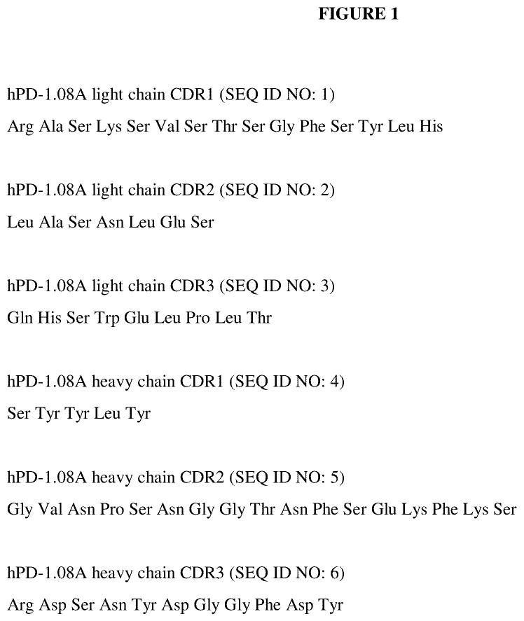

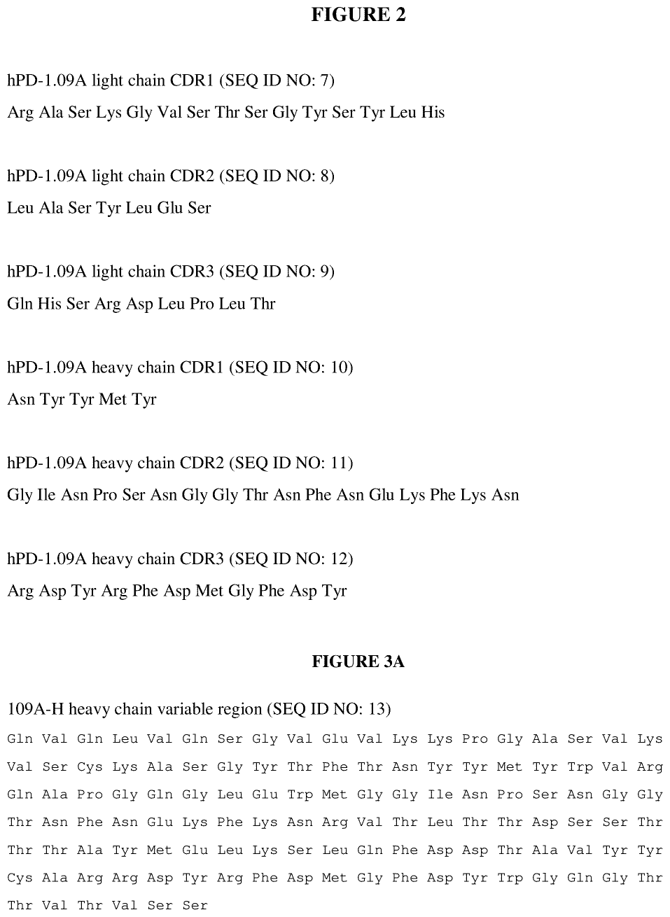

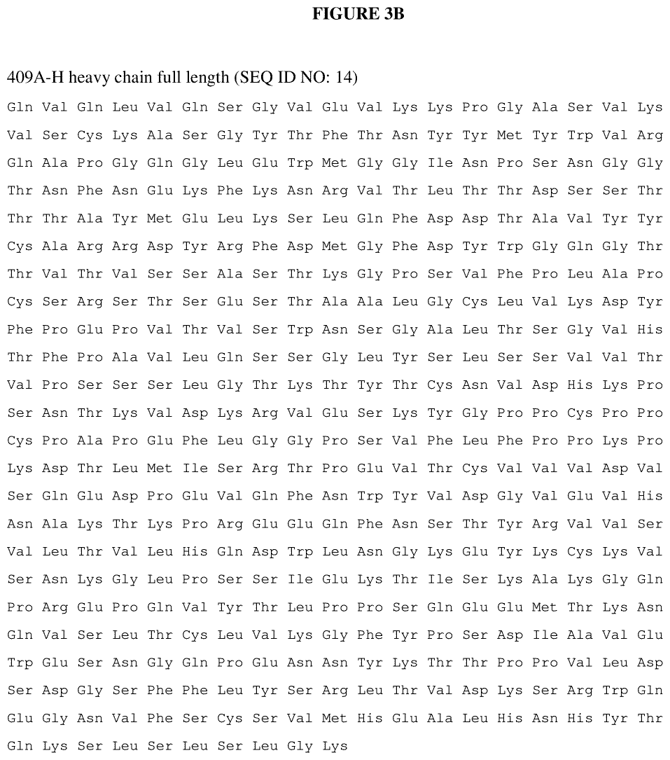

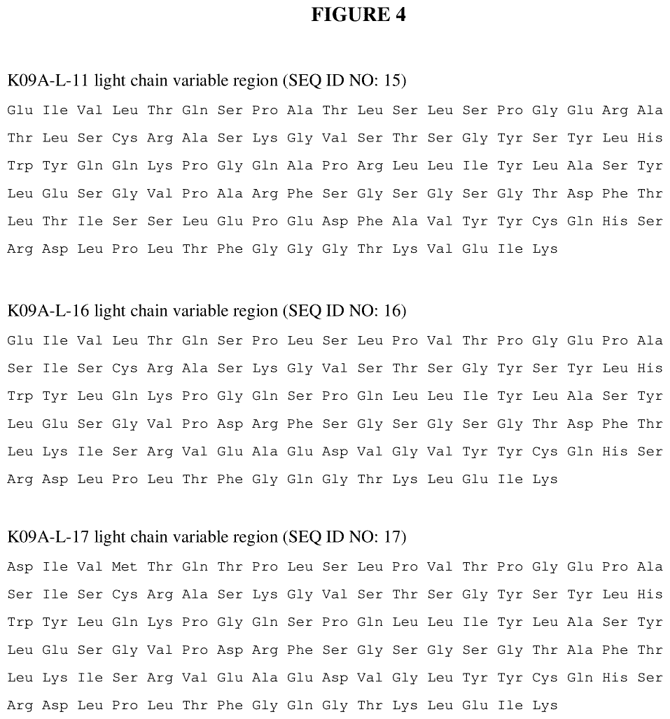

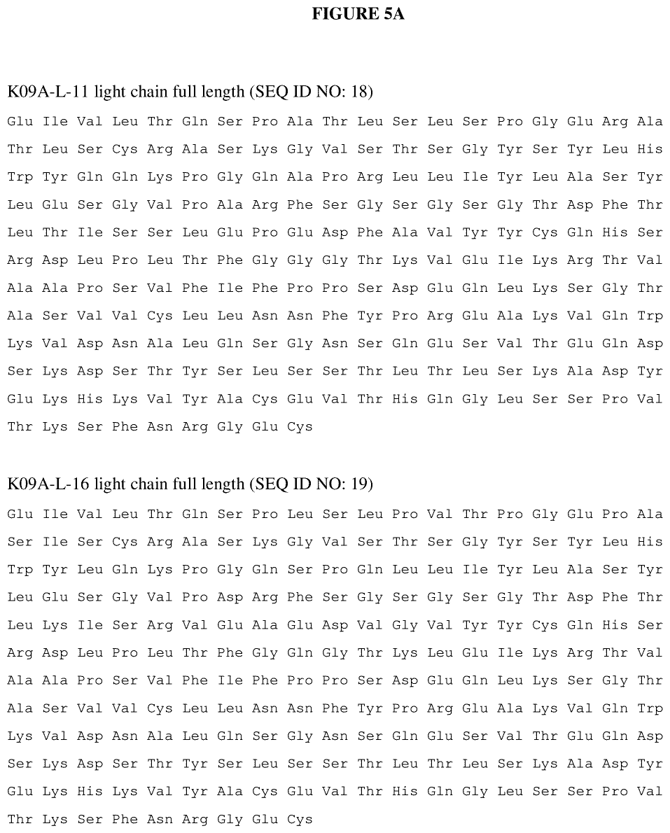

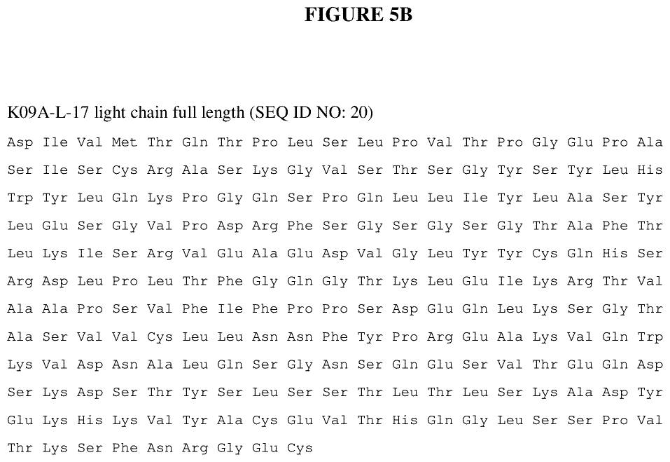

Table 2 below provides a list of the amino acid sequences of exemplary anti-PD-1 mAbs for use in the treatment methods, medicaments and disclosed uses, and the sequences are shown in FIGS. 1-5B.

TABLE 2
EXEMPLARY ANTI-HUMAN PD-1 MONOCLONAL ANTIBODIES
A. Comprises light and heavy chain CDRs
of hPD-1.08A in WO2008/156712

| | |
|---|---|
| CDRL1 | SEQ ID NO: 1 |
| CDRL2 | SEQ ID NO: 2 |
| CDRL3 | SEQ ID NO: 3 |
| CDRH1 | SEQ ID NO: 4 |
| CDRH2 | SEQ ID NO: 5 |
| CDRH3 | SEQ ID NO: 6 |

B. Comprises light and heavy chain CDRs of
hPD-1.09A in WO2008/156712

| | |
|---|---|
| CDRL1 | SEQ ID NO: 7 |
| CDRL2 | SEQ ID NO: 8 |
| CDRL3 | SEQ ID NO: 9 |
| CDRH1 | SEQ ID NO: 10 |
| CDRH2 | SEQ ID NO: 11 |
| CDRH3 | SEQ ID NO: 12 |

C. Comprises the mature h109A heavy chain
variable region and one of the mature K09A
light chain variable regions in WO2008/156712

| | |
|---|---|
| Heavy chain VR | SEQ ID NO: 13 |
| Light chain VR | SEQ ID NO: 15 or SEQ ID NO: 16 or |
| | SEQ ID NO: 17 |

D. Comprises the mature 409 heavy chain and one of
the mature K09A light chains in WO2008/156712

| | |
|---|---|
| Heavy chain | SEQ ID NO: 14 |
| Light chain | SEQ ID NO: 18 or SEQ ID NO: 19 or |
| | SEQ ID NO: 20 |

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by positron emission tomography (PET) imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and realtime quantitative RT-PCR.

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≥5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

The level of PD-L mRNA expression may be compared to the mRNA expression levels of one or more reference genes that are frequently used in quantitative RT-PCR, such as ubiquitin C.

In some instances, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue (i.e. non-malignant tissue). PD-L1 expression in a tumor sample is preferably determined to be elevated if PD-L1 protein (and/or PD-L1 mRNA) in the sample is at least 10%, 20%, or 30% greater than in the control.

A “pembrolizumab biosimilar” means a biological product manufactured by an entity other than Merck & Co., Inc. d.b.a. Merck Sharp and Dohme (MSD) and which is approved by a regulatory agency in any country for marketing as a pembrolizumab biosimilar. A pembrolizumab biosimilar may include as the drug substance a pembrolizumab variant or an antibody with the same amino acid sequence as pembrolizumab.

As used herein, a “pembrolizumab variant” means a monoclonal antibody which comprises heavy chain and light chain sequences that are identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g, the variant positions are located in the FR regions and/or the constant region. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Responsder patient” when referring to a specific anti-tumor response to treatment with a combination therapy described herein, means the patient exhibited the anti-tumor response.

“Sustained response” means a sustained therapeutic effect after cessation of treatment with a therapeutic agent, or a combination therapy described herein. In some instances, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer a combination therapy of a PD-1 antagonist and a multi-RTK inhibitor to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50: 1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some instances, response to a combination therapy described herein is assessed using RECIST 1.1 criteria or irRC (bidimensional or unidimensional) and the treatment achieved by a combination of a multi-RTK inhibitor and a PD-1 antagonist is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some instances, response to a combination of a multi-RTK inhibitor and a PD-1 antagonist is any of PR, CR, PFS, DFS, OR and OS that is assessed using RECIST 1.1 response criteria. The treatment regimen for the disclosed combination that is effective to treat a cancer patient may vary according to factors such as the disease state, age, and weight of the patient, and the ability of the therapy to elicit an anti-cancer response in the subject. The treatment methods, medicaments, and disclosed uses may not be effective in achieving a positive therapeutic effect in every subject, they should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of a multi-RTK inhibitor and a PD-1 antagonist.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood or heme cancers) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in Nishino M, Giobbie-Hurder A, Gargano M, Suda M, Ramaiya N H, Hodi F S. “Developing a Common Language for Tumor Response to Immunotherapy: Immune-related Response Criteria using Unidimensional measurements,” Clin Cancer Res. 2013, 19 (14): 3936-3943). These criteria utilize the longest diameter (cm) of each lesion.

“Variable regions” or “V region” as used herein means the segment of IgG chains which is variable in sequence between different antibodies. It extends to Kabat residue 109 in the light chain and 113 in the heavy chain.

“Multi-RTK Inhibitor” means a small molecule compound that inhibits the kinase activities of at least each of the following RTKs: (i) VEGFR2, (ii) at least one FGFR selected from the group consisting of FGFR1, 2, 3 and 4; and (iii) RET. The multi-RTK inhibitor may also inhibit the kinase activities of VEGFR1, VEGFR3, fibroblast growth factor (FGF) receptors FGFR1, 2, 3 and 4, platelet-derived growth factor (PDGF) receptor alpha (PDGFRα); and KIT. The multi-RTK inhibitor may have the structure represented by Formula (I):

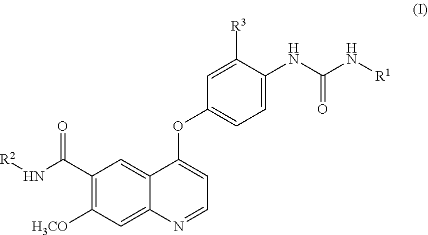

wherein R¹ is C₁₋₆ alkyl or C₃₋₈ cycloalkyl, R² is a hydrogen atom or C₁₋₆ alkoxy, and R³ is a hydrogen atom or a halogen atom.

The treatment methods, medicaments and disclosed uses provide for the multi-RTK inhibitor as the compound, of the following structure:

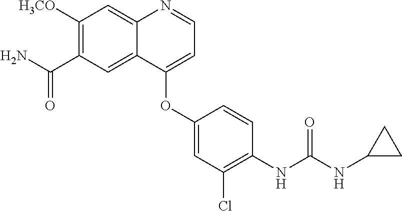 [SMILES: COc1cc2nccc(Oc3ccc(NC(=O)NC4CC4)c(Cl)c3)c2cc1C(N)=O]

which is known as lenvatinib, or a pharmaceutically acceptable salt thereof (e.g. lenvatinib mesilate).

### II. METHODS, USES AND MEDICAMENTS

In one aspect, a method for treating a cancer in an individual is provided that comprises administering to the individual a combination therapy which comprises a PD-1 antagonist and a multi-RTK inhibitor.

The combination therapy may also comprise one or more additional therapeutic agents. The additional therapeutic agent may be, e.g., a chemotherapeutic other than a multi-RTK inhibitor, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (such as bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, preferrably calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), cpirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; ctoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (such as T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy disclosed herein may be administered either alone or in a medicament (also referred to herein as a pharmaceutical composition) which comprises the therapeutic agent and one or more pharmaceutically acceptable carriers, excipients and diluents, according to standard pharmaceutical practice. Each therapeutic agent may be prepared by formulating a compound or pharmaceutically acceptable salt thereof represented by Formula (I), and an anti-PD-1 antibody separately, and the both may be administered either at the same time or separately. Further, the two formulations may be placed in a single package, to provide the so called kit formulation. In some configurations, both compounds may be contained in a single formulation.

The compound or pharmaceutically acceptable salt thereof represented by Formula (I) can be produced by the method described in Reference 17. Examples of the pharmaceutically acceptable salt include salts with inorganic acids, salts with organic acids, salts with inorganic bases, salts with organic bases, and salts with acidic or basic amino acids. Preferred examples of the salts with inorganic acids include salts with hydrochloric acid, hydrobromic acid, sulfuric acid, nitric acid, phosphoric acid and the like. Preferred examples of the salts with organic acids include salts with acetic acid, succinic acid, fumaric acid, maleic acid, tartaric acid, citric acid, lactic acid, stearic acid, benzoic acid, methanesulfonic acid, ethanesulfonic acid, p-toluenesulfonic acid and the like. Preferred examples of the salts with inorganic bases include alkaline metal salts such as a sodium salt and a potassium salt; alkaline earth metal salts such as a calcium salt and a magnesium salt; an aluminum salt; and an ammonium salt. Preferred examples of the salts with organic bases include salts with diethylamine, diethanolamine, meglumine, N,N-dibenzylethylenediamine and the like. Preferred examples of the salts with acidic amino acids include salts with aspartic acid, glutamic acid and the like. Preferred examples of the salts with basic amino acids include salts with arginine, lysine, ornithine and the like. More preferred pharmaceutically acceptable salts are salts with organic acids and especially preferred pharmaceutically acceptable salts are salts with methanesulfonic acid.

Each therapeutic agent in a combination therapy disclosed herein may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some instances, the multi-RTK inhibitor is administered before administration of the PD-1 antagonist, while in other instances, the multi-RTK inhibitor is administered after administration of the PD-1 antagonist.

In some instances, at least one of the therapeutic agents in the combination therapy is administered using the same dosage regimen (dose, frequency and duration of treatment) that is typically employed when the agent is used as monotherapy for treating the same cancer. In other instances, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy disclosed herein can be administered orally in the form of a solid formulation such as a tablet, granule, fine granule, powder or capsule, or in the form of a liquid, jelly, syrup, or the like. Each small molecule therapeutic agent in a combination therapy disclosed herein may be administered parenterally, including the intravenous, intramuscular, intraperitoneal, subcutaneous, rectal, topical, and transdermal routes of administration.

A combination therapy disclosed herein may be used prior to or following surgery to remove a tumor and may be used prior to, during or after radiation therapy.

In some instances, a combination therapy disclosed herein is administered to a patient who has not been previously treated with a biotherapeutic or chemotherapeutic agent, i.e., is treatment-naïve. In other instances, the combination therapy is administered to a patient who failed to achieve a sustained response after prior therapy with a biotherapeutic or chemotherapeutic agent, i.e., is treatment-experienced.

A combination therapy disclosed herein is typically used to treat a tumor that is large enough to be found by palpation or by imaging techniques well known in the art, such as magnetic resonance imaging (MRI), ultrasound, or computerized axial tomography (CAT) scan.

A combination therapy disclosed herein is preferably administered to a human patient who has a cancer that tests positive for PD-L1 expression. PD-L1 expression is detected preferably using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 antagonist and the multi-RTK inhibitor, but it is envisioned that the physician could order the first or subsequent diagnostic tests at any time after initiation of treatment, such as for example after completion of a treatment cycle.

Selecting a dosage regimen (also referred to herein as an administration regimen) for a combination therapy disclosed herein depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) ANTIBODY THERAPY, Bios Scientific Pub. Ltd, Oxfordshire, UK; Kresina (ed.) (1991) MONOCLONAL ANTIBODIES, CYTOKINES AND ARTHRITIS, Marcel Dekker, New York, NY; Bach (ed.) (1993) MONOCLONAL ANTIBODIES AND PEPTIDE THERAPY IN AUTOIMMUNE DISEASES, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; PHYSICIANS' DESK REFERENCE 2003 (Physicians' Desk Reference, 57th Ed.); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents in a combination therapy disclosed herein may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

The dose of the compound or pharmaceutically acceptable salt thereof represented by Formula (I) may be appropriately selected depending on the degrees of symptoms, age, sex, and body weight of the patient, difference in sensitivity, route, time and interval of administration, type of pharmaceutical formulation, and/or the like. Typically, in cases where oral administration is carried out for an adult (60 kg body weight), the dose is 1 to 600 mg, preferably 5 to 400 mg, more preferably 5 to 200 mg per day. The dose may be administered at one time or divided into smaller doses provided 2 to 3 times per day.

In some instances that employ an anti-human PD-1 mAb as the PD-1 antagonist in the combination therapy, the dosing regimen will comprise administering the anti-human PD-1 mAb at a dose of 1, 2, 3, 5 or 10 mg/kg at intervals of about 14 days (±2 days) or about 21 days (±2 days) or about 30 days (±2 days) throughout the course of treatment. The dosage of an anti-PD-1 antibody can be appropriately selected in the same manner as above. Typically, in cases where intravenous administration is carried out for an adult (60 kg body weight), the dose is 2 mg/kg on a schedule of once every 3 weeks on a 6-week cycle (a total of 2 doses). The antibody is administered for 1 to 10 cycles at an appropriate interval.

In other instances that employ an anti-human PD-1 mAb as the PD-1 antagonist in the combination therapy, the dosing regimen will comprise administering the anti-human PD-1 mAb at a dose of from about 0.005 mg/kg to about 10 mg/kg, with intra-patient dose escalation. The interval between doses can be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In specific instances, a subject will be administered an intravenous (IV) infusion of a medicament comprising any of the PD-1 antagonists described herein.

The PD-1 antagonist in the combination therapy is preferably nivolumab in some instances, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W.

The PD-1 antagonist in the combination therapy preferably is pembrolizumab, a pembrolizumab variant or a pembrolizumab biosimilar in some instances, which is administered in a liquid medicament at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, 10 mg Q3W and flat-dose equivalents of any of these doses, i.e., such as 200 mg Q3W. In some instances, pembrolizumab is provided as a liquid medicament which comprises 25 mg/ml pembrolizumab, 7% (w/v) sucrose, 0.02% (w/v) polysorbate 80 in 10 mM histidine buffer pH 5.5.

In some instances, the selected dose of pembrolizumab is administered by IV infusion over a time period of between 25 and 40 minutes, or about 30 minutes.

The optimal dose for pembrolizumab in combination with lenvatinib or a pharmaceutically acceptable salt thereof (e.g. lenvatinib mesilate) may be identified by dose escalation or dose de-escalation of one or both of these agents. In some instances, the combination therapy comprises a 21 day treatment cycle in which pembrolizumab is administered at 200 mg Q3W by IV and lenvatinib mesilate is administered at (a) 24 mg per day orally, (b) 20 mg per day orally or (c) 14 mg per day orally, each as lenvatinib. In an embodiment, a patient is treated first with 200 mg of pembrolizumab Q3W by IV and 24 mg (as lenvatinib) of lenvatinib mesilate per day orally until at least one DLT is observed and then the dosage of lenvatinib mesilate is reduced to 20 or 14 mg (each as lenvatinib) per day while the pembrolizumab dose is continued at 200 mg of pembrolizumab Q3W.

As an example dosing regimen, lenvatinib or a pharmaceutically acceptable salt thereof can be administered with water orally once a day, with or without food, in 21 day cycles at approximately the same time each day. Lenvatinib or a pharmaceutically acceptable salt thereof can be provided as 4 mg and 10 mg (each as lenvatinib) capsules. On Day one (D1) of each cycle, lenvatinib or a pharmaceutically acceptable salt thereof can be administered approximately within 1 hour after completion of pembrolizumab administration. Pembrolizumab may be provided as a sterile, preservative-free, white to off-white lyophilized powder in single-use vials. Each vial can be reconstituted and diluted for intravenous infusion. Each 2 mL of reconstituted solution may contain approximately 50 mg of prembrolizumab. In some instances, pembrolizumab may be provided as a sterile, preservative-free, clear to slightly opalescent, colorless to slightly yellow solution that requires dilution for intravenous infusion. Each vial may contain 100 mg of pembrolizumab in 4 mL of solution. Each 1 mL of solution may contain 25 mg of pembrolizumab. Pembrolizumab may be administered as a dose of 200 mg as a 30-minute intravenous infusion, Q3W (25 minutes to 40 minutes, for example).

In cases where an oral solid formulation is prepared, a pharmaceutically acceptable vehicle, and, as required, a binder, disintegrator, lubricant, coloring agent, flavoring agent and/or the like may be added to the principal component, that is, a compound or pharmaceutically acceptable salt thereof represented by Formula (I), and an anti-PD-1 antibody, to prepare, thereafter, a tablet, granule, fine granule, powder, capsule or the like according to a conventional method. Examples of the vehicle include lactose, corn starch, white soft sugar, glucose, sorbitol, crystalline cellulose and silicon dioxide. Examples of the binder include polyvinyl alcohol, ethylcellulose, methylcellulose, gum Arabic, hydroxypropylcellulose and hydroxypropylmethylcellulose. Examples of the lubricant include magnesium stearate, talc, and silica. Examples of the coloring agent include titanium oxide, iron sesquioxide, yellow iron sesquioxide, cochineal, carmine, and riboflavin. Examples of the flavoring agent include cocoa powder, ascorbic acid, tartaric acid, peppermint oil, borneol, and cinnamon powder. These tablets and granules may be coated as may be required.

In some instances, the patient is treated with the combination therapy for at least 24 weeks, e.g., eight 3-week cycles. In some instances, treatment with the combination therapy continues until the patient exhibits evidence of PD or a CR.

In some instances, the patient selected for treatment with the combination therapy disclosed herein if the patient has been diagnosed with NSCLC, RCC, endometrial cancer, urothelial cancer, squamous cell carcinoma of head and neck or melanoma.

A medicament is provided which comprises a PD-1 antagonist as described above and a pharmaceutically acceptable excipient. When the PD-1 antagonist is a biotherapeutic agent, e.g., a mAb, the antagonist may be produced in CHO cells using conventional cell culture and recovery/purification technologies.

In some instances, a medicament comprising an anti-PD-1 antibody as the PD-1 antagonist may be provided as a liquid formulation or prepared by reconstituting a lyophilized powder with sterile water for injection prior to use. For example, WO 2012/135408 describes the preparation of liquid and lyophilized medicaments comprising pembrolizumab that are suitable for any treatment methods, medicaments, and disclosed uses. In some instances, a medicament comprising pembrolizumab is provided in a glass vial which contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, United States Pharmacopeial (USP) Convention. The solution requires dilution for IV infusion.

In cases where an injection is prepared, a pH adjustor, buffering agent, suspending agent, solubilizer, stabilizer, isotonic agent, preservative and/or the like may be added as required to the principal component, to prepare an intravenous, subcutaneous or intramuscular injection, or an intravenous drip infusion. As required, these may be prepared into lyophilized products by conventional methods. Examples of the suspending agent include methylcellulose, polysorbate 80, hydroxyethylcellulose, gum Arabic, powdered tragacanth, sodium carboxymethylcellulose, and polyoxyethylene sorbitan monolaurate. Examples of the solubilizer include polyoxyethylene hydrogenated castor oil, polysorbate 80, nicotinamide, polyoxyethylene sorbitan monolaurate, macrogol, and glycerin fatty acid ester. Examples of the stabilizer include sodium sulfite and sodium metabisulfite. Examples of the preservative include methyl parahydroxybenzoate, ethyl parahydroxybenzoate, sorbic acid, phenol, cresol, and chlorocresol.

A medicament is provided which comprises lenvatinib or a pharmaceutically acceptable salt thereof (e.g. lenvatinib mesilate) and a pharmaceutically acceptable excipient. In some instances, lenvatinib or a pharmaceutically acceptable salt thereof (e.g. lenvatinib mesilate) is provided as a 4 mg or 10 mg (each as lenvatinib) capsule and formulated with calcium carbonate, mannitol, microcrystalline cellulose, hydroxypropylcellulose, low-substituted hydroxypropylcellulose, and talc.

The PD-1 antagonist and lenvatinib or a pharmaceutically acceptable salt thereof (e.g. lenvatinib mesilate) medicaments described herein may be provided as a kit which comprises a first container and a second container and a package insert. The first container contains at least one dose of a medicament comprising a PD-1 antagonist, the second container contains at least one dose of a medicament comprising a multi-RTK inhibitor, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The kit may preferably provide for the PD-1 antagonist to be an anti-PD-1 antibody and the instructions may state that the medicaments are intended for use in treating a patient having a cancer that tests positive for PD-L1 expression by an IHC assay.

These and other aspects disclosed herein, including the exemplary specific treatment methods, medicaments, and uses listed below, will be apparent from the teachings contained herein.

### Specific Treatment Methods, Medicaments, and Uses

1. A method for treating a cancer in an individual comprising administering to the individual a combination therapy which comprises a PD-1 antagonist and a multi-RTK inhibitor.
2. The method of embodiment 1, wherein the PD-1 antagonist is a monoclonal antibody, or an antigen binding fragment thereof.
3. The method of embodiment 1 or 2, wherein the multi-RTK inhibitor is lenvatinib or a pharmaceutically acceptable salt thereof, and the PD-1 antagonist is not MPDL3280A.
4. A medicament comprising a PD-1 antagonist, which is not MPDL3280A, for use in combination with a multi-RTK inhibitor for treating a cancer in an individual, wherein the PD-1 antagonist is a monoclonal antibody, or an antigen binding fragment thereof.
5. A medicament comprising a multi-RTK inhibitor for use in combination with a PD-1 antagonist, which is not MPDL3280A, for treating a cancer in an individual.
6. The medicament of embodiment 4 or 5, which further comprises a pharmaceutically acceptable excipient.
7. Use of a PD-1 antagonist, which is not MPDL3280A, in the manufacture of medicament for treating a cancer in an individual when administered in combination with a multi-RTK inhibitor.
6. Use of a multi-RTK inhibitor in the manufacture of a medicament for treating a cancer in an individual when administered in combination with a PD-1 antagonist, which is not MPDL3280A.
7. Use of a PD-1 antagonist, which is not MPDL3280A, and a multi-RTK inhibitor in the manufacture of medicaments for treating a cancer in an individual.
8. A kit which comprises a first container, a second container and a package insert, wherein the first container comprises at least one dose of a medicament comprising an anti-PD-1 antagonist, which is not MPDL3280A, the second container comprises at least one dose of a medicament comprising a multi-RTK inhibitor, and the package insert comprises instructions for treating an individual for cancer using the medicaments.
9. The kit of embodiment 8, wherein the instructions state that the medicaments are intended for use in treating an individual having a cancer that tests positive for PD-L1 expression by an immunohistochemical (IHC) assay.
10. The method, medicament, use or kit of any of embodiments 1 to 9, wherein the individual is a human and the PD-1 antagonist is a monoclonal antibody, or an antigen binding fragment thereof, which specifically binds to human PD-L1 and blocks the binding of human PD-L1 to human PD-1.
11. The method, medicament, use or kit of embodiment 9, wherein the PD-1 antagonist is BMS-936559, MEDI4736, or MSB0010718C.
12. The method, medicament, use or kit of any of embodiments 1 to 9, wherein the individual is a human, and the PD-1 antagonist is a monoclonal antibody, or an antigen binding fragment thereof, which specifically binds to human PD-1 and blocks the binding of human PD-L1 to human PD-1.
13. The method, medicament, use or kit of embodiment 12, wherein the PD-1 antagonist also blocks binding of human PD-L2 to human PD-1.
14. The method, medicament, use or kit of embodiment 13, wherein the monoclonal antibody, or antigen binding fragment thereof, comprises: (a) light chain CDRs of SEQ ID NOs: 1, 2 and 3 and heavy chain CDRs of SEQ ID NOs: 4, 5 and 6; or (b) light chain CDRs of SEQ ID NOs: 7, 8 and 9 and heavy chain CDRs of SEQ ID NOs: 10, 11 and 12.
15. The method, medicament, use or kit of embodiment 13, wherein the monoclonal antibody, or antigen binding fragment thereof, comprises light chain CDRs of SEQ ID NOs: 7, 8 and 9 and heavy chain CDRs of SEQ ID NOs: 10, 11 and 12.
16. The method, medicament, use or kit of embodiment 13, wherein the PD-1 antagonist is an anti-PD-1 monoclonal antibody which comprises a heavy chain and a light chain, and wherein the heavy chain comprises SEQ ID NO:21 and the light chain comprises SEQ ID NO:22.
17. The method, medicament, use or kit of embodiment 13, wherein the PD-1 antagonist is an anti-PD-1 monoclonal antibody which comprises a heavy chain and a light chain, and wherein the heavy chain comprises SEQ ID NO:23 and the light chain comprises SEQ ID NO:24.
18. The method, medicament, use or kit of any of embodiments 10-17, wherein the cancer is a solid tumor.
19. The method, medicament, use or kit of any of embodiments 10-17, wherein the cancer is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell carcinoma (RCC), small-cell lung cancer (SCLC) or triple negative breast cancer.
20. The method, medicament, use or kit of any of embodiments 10-17, wherein the cancer is NSCLC, RCC, endometrial cancer, urothelial cancer, squamous cell carcinoma of head and neck or melanoma.
21. The method, medicament, use or kit of any of embodiments 10-17, wherein the individual has not been previously treated for NSCLC, RCC, endometrial cancer, urothelial cancer, squamous cell carcinoma of head and neck or melanoma.
23. The method, medicament, use or kit of any of embodiments 10-17, wherein the cancer is acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), or small lymphocytic lymphoma (SLL).
24. The method, medicament, use or kit of any of embodiments 10-23, the cancer tests positive for human PD-L1.
25. The method, medicament, use or kit of embodiment 24, wherein the human PD-L1 expression is elevated.
26. The method, medicament, use or kit of embodiment 13, wherein the PD-1 antagonist is pembrolizumab, a pembrolizumab variant, a pembrolizumab biosimilar or nivolumab.
27. The method, medicament, use or kit of embodiment 26, wherein pembrolizumab is formulated as a liquid medicament which comprises 25 mg/ml pembrolizumab, 7% (w/v) sucrose, 0.02% (w/v) polysorbate 80 in 10 mM histidine buffer pH 5.5.
28. The method, medicament, use or kit of any of embodiments 1 to 27, wherein the mutli-RTK inhibitor is lenvatinib or a pharmaceutically acceptable salt thereof (e.g. lanvatinib mesilate).
29. The method, medicament, use or kit of any of embodiments 1 to 28, wherein the mutli-RTK inhibitor is lenvatinib mesilate and is formulated with calcium carbonate, mannitol, microcrystalline cellulose, hydroxypropylcellulose, low-substituted hydroxypropylcellulose, and talc.
30. A method for treating a human individual diagnosed with a cancer, comprising administering to the individual a combination therapy which comprises pembrolizumab and lenvatinib or a pharmaceutically acceptable salt thereof (e.g. lanvatinib mesilate), wherein lenvatinib or a pharmaceutically acceptable salt thereof (e.g. lanvatinib mesilate) is administered at a daily dose of 24 mg, 20 mg or 14 mg, each as lenvatinib, and pembrolizumab is administered at 200 mg Q3W.
31. A medicament comprising pembrolizumab for use in combination with lenvatinib or a pharmaceutically acceptable salt thereof (e.g. lanvatinib mesilate) for treating a cancer in a human individual by a method comprising administering to the individual lenvatinib or a pharmaceutically acceptable salt thereof (e.g. lanvatinib mesilate) at a daily dose of 24 mg, 20 mg or 14 mg, each as lenvatinib, and pembrolizumab at 200 mg Q3W.
32. A medicament comprising lenvatinib or a pharmaceutically acceptable salt thereof (e.g. lanvatinib mesilate) for use in combination with pembrolizumab for treating a cancer in a human individual by a method comprising administering to the individual lenvatinib or a pharmaceutically acceptable salt thereof (e.g. lanvatinib mesilate) at a daily dose of 24 mg, 20 mg or 14 mg, each as lenvatinib, and pembrolizumab at 200 mg Q3W.
33. The method or medicament of any of embodiments 30 to 32, wherein the cancer is NSCLC, RCC, endometrial cancer, urothelial cancer, squamous cell carcinoma of head and neck or melanoma.
34. The method or medicament of embodiment 33, wherein the individual has not been previously treated for NSCLC, RCC, endometrial cancer, urothelial cancer, squamous cell carcinoma of head and neck or melanoma.
35. The method or medicament of any of embodiments 31 to 34, wherein a tissue section of the cancer removed from the individual prior to administration of the combination therapy tested positive for PD-L1 expression.
36. The method or medicament of embodiment 35, wherein at least 50% of the tumor cells in the tissue section tested positive for PD-L1 expression by an immunohistochemical (IHC) assay.
37. The method or medicament of embodiment 36, wherein the IHC assay employed the antibody 22C₃ to detect PD-L1 expression.
38. The method or medicament of any of embodiments 31 to 37, wherein pembrolizumab is administered by IV infusion for 25 to 40 minutes or about 30 minutes.

### General Methods

Standard methods in molecular biology are described Sambrook, Fritsch and Maniatis (1982 & 1989 2^{nd} Edition, 2001 3^{rd} Edition) MOLECULAR CLONING, A LABORATORY MANUAL, Cold Spring Harbor Laboratory Press, Cold Spring Harbor, NY; Sambrook and Russell (2001) MOLECULAR CLONING, 3^{rd} ed., Cold Spring Harbor Laboratory Press, Cold Spring Harbor, NY; Wu (1993) RECOMBINANT DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) CURRENT PROTOCOLS IN PROTEIN SCIENCE, Vol. 1, John Wiley and Sons, Inc., New York). Chemical analysis, chemical modification, post-translational modification, production of fusion proteins, glycosylation of proteins are described (see, e.g., Coligan, et al. (2000) Current PROTOCOLS IN PROTEIN SCIENCE, Vol. 2, John Wiley and Sons, Inc., New York; Ausubel, et al. (2001) CURRENT PROTOCOLS IN MOLECULAR BIOLOGY, Vol. 3, John Wiley and Sons, Inc., NY, NY, pp. 16.0.5-16.22.17; Sigma-Aldrich, Co. (2001) PRODUCTS FOR LIFE SCIENCE RESEARCH, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) CURRENT PROTOCOLS IN IMMUNOLOGY, Vol. 1, John Wiley and Sons, Inc., New York; Harlow and Lane (1999) USING ANTIBODIES, Cold Spring Harbor Laboratory Press, Cold Spring Harbor, NY; Harlow and Lane, supra). Standard techniques for characterizing ligand/receptor interactions are available (see, e.g., Coligan, et al. (2001) CURRENT PROTOCOLS IN IMMUNOLOGY, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16:741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Table 3 provides a brief description of the sequences in the sequence listing.

TABLE 3

| SEQ ID NO: | Description |
|---|---|
| 1 | hPD-1.08A light chain CDR1 |
| 2 | hPD-1.08A light chain CDR2 |
| 3 | hPD-1-08A light chain CDR3 |
| 4 | hPD-1.08A heavy chain CDR1 |
| 5 | hPD-1.08A heavy chain CDR2 |
| 6 | hPD-1.08A heavy chain CDR3 |
| 7 | hPD-1.09A light chain CDR1 |
| 8 | hPD-1.09A light chain CDR2 |
| 9 | hPD-1.09A light chain CDR3 |
| 10 | hPD-1.09A heavy chain CDR1 |
| 11 | hPD-1.09A heavy chain CDR2 |
| 12 | hPD-1.09A heavy chain CDR3 |
| 13 | 109A-H heavy chain variable region |
| 14 | 409A-H heavy chain full length |
| 15 | K09A-L-11 light chain variable region |
| 16 | K09A-L-16 light chain variable region |
| 17 | K09A-L-17 light chain variable region |
| 18 | K09A-L-11 light chain full length |
| 19 | K09A-L-16 light chain full length |
| 20 | K09A-L-17 light chain full length |
| 21 | Pembrolizumab Heavy chain |
| 22 | Pembrolizumab Light chain |
| 23 | Nivolumab Heavy chain |
| 24 | Nivolumab light chain |
| 25 | Human PD-L1 |

### III. EXAMPLES

### Example 1: Anti-Tumor Effect by Administration of Lenvatinib and Anti-PD-1 Antibody

A DMEM culture medium (high-glucose type) containing 10% fetal bovine serum (FBS) and penicillin/streptomycin was used to culture a mouse lung cancer cell line LL/2 (LLc1) (ATCC number: CRL-1642). Next, phosphate buffered saline (PBS) was used to prepare a cell suspension having a concentration of 2.0×10⁷ cells/mL. The cell suspension was subcutaneously transplanted at a dose of 0.1 mL on the right lateral side of the body of each of 7-week-old mice (C57BL/6J, female, Charles River Laboratories Japan Inc.). Eight (8) days after the transplantation, an electronic digital caliper (Digimatic (TM) Caliper; Mitutoyo Corporation) was used to measure the short and long diameters of a tumor of interest. The following equation was used to calculate the tumor volume TV.

Tumor Volume TV (mm³)=Long Diameter (mm)×Short Diameter (mm)×Short Diameter (mm)/2.

Based on the tumor volumes on the first day of administration, grouping was carried out such that the average values of the tumor volumes were almost the same. A 1 mg/ml solution of lenvatinib was prepared using water for injection (Otsuka Pharmaceutical Co., Ltd.) and was orally administered at a dose of 0.2 mL/20 g mouse body weight once daily for 14 days. 0.2 mL of an administration sample containing 2.5 mg/mL of an anti-mouse-PD-1 antibody (Clone: RMP1-14, BioXCell, Catalog #: BE0146), which had been diluted with PBS, was intraperitoneally administered (at a dosage of 500 μg/head) once every 3 days a total of 5 times (day 1, day 4, day 7, day 10, and day 13, with the day of the grouping set to day 1). To the control group, Otsuka water for injection was orally administered at a dose of 0.2 mL/20 g mouse body weight once daily for 14 days. Each group including 5 mice was used to conduct the experiment. The day (day 15) after the final administration, the respective tumor volumes (TV) were determined for the control group, the lenvatinib administration group, the anti-mouse-PD-1 antibody administration group, and the combination administration group. The values obtained by logarithmically transforming the tumor volumes were used to carry out statistical analysis.

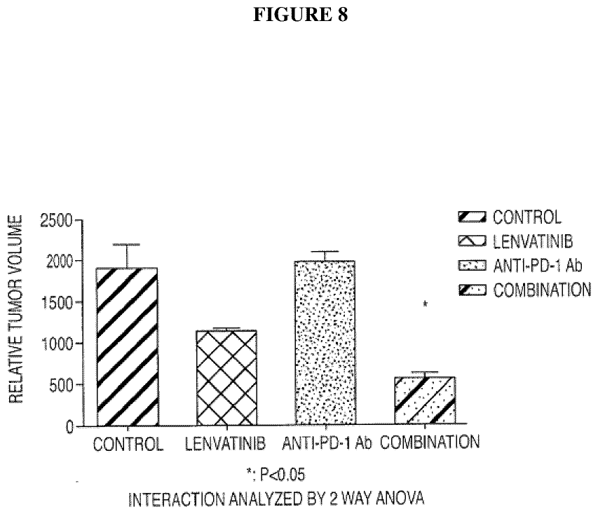

In the subcutaneous LL/2 (LLc1) transplantation model, the combination of lenvatinib and the anti-mouse-PD-1 antibody exhibited a significantly higher anti-tumor effect than either administered alone. For example, at day 15, the combination group had greater than two and half fold less tumor volume compared to the control group and the anti-PD-1 group and over one and a half fold less tumor volume compared to the lenvatinib group. The daily change in the tumor volume is shown in Table 4. In addition, the tumor volumes the day after the final administration are shown in FIG. 8.

TABLE 4

| | Day 1 | Day 4 | Day 8 | Day 10 | Day 13 | Day15 |
|---|---|---|---|---|---|---|
| Control group | 101 | 273 | 564 | 852 | 1455 | 1910 |
| Lenvatinib group | 100 | 243 | 394 | 553 | 837 | 1143 |
| Anti-PD-1 antibody | 100 | 207 | 442 | 682 | 1083 | 1984 |
| group | | | | | | |
| Combination group | 100 | 203 | 351 | 452 | 573 | 691 |

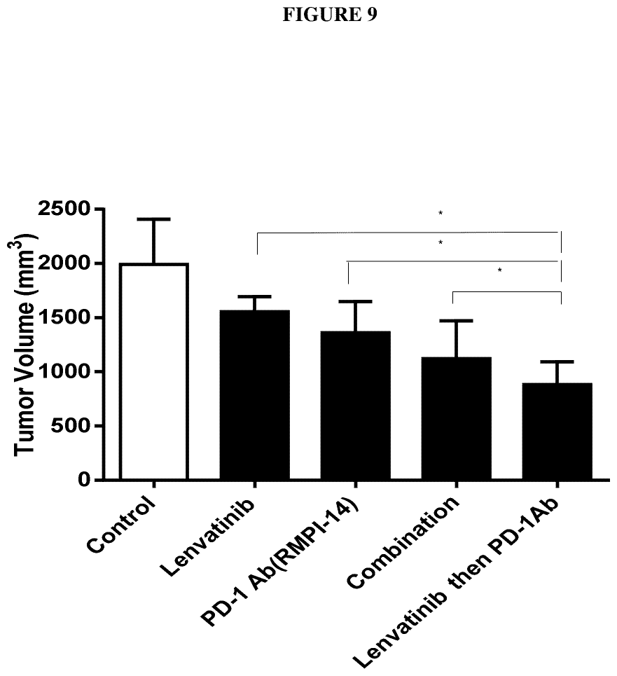

FIG. 9 shows the anti-cancer or -tumor effect on the eleventh day since commencing treatment in a mouse model with colon cancer. A RPMI1640 culture medium containing 10% FBS and penicillin/streptomycin was used to culture a mouse colon carcinoma cell line CT26.WT (ATCC number: CRL-2638). Hank's Balanced Salt Solution (HBSS) was used to prepare a cell suspension having a concentration of 3.0×10⁷ cells/mL. The cell suspension was subcutaneously transplanted at a dose of 0.1 mL on the right lateral side of the body of each of 7-week-old mice (BALB/cAnNCrlCrlj, female, Charles River Laboratories Japan Inc.). 8 days after the transplantation, an electronic digital caliper (Digimatic (TM) Caliper; Mitutoyo Corporation) was used to measure the short and long diameters of a tumor of interest. The following equation was used to calculate the tumor volume TV.

$$Tumor Volume TV ( {mm}^{3} ) =$$

$$Long Diameter ( mm ) \times Short Diameter ( mm ) \times Short Diameter$$

$$( mm ) / 2.$$

Based on the tumor volumes on the first day of administration, grouping was carried out such that the average values of the tumor volumes were almost the same. For each of lenvatinib only treatment group, simultaneous combination treatment group, and lenvatinib then PD-1 Ab group, 1 mg/ml solution of lenvatinib mesilate was prepared using water for injection (Otsuka Pharmaceutical Co., Ltd.) and was orally administered at a dose of 0.2 mL/20 g mouse body weight once daily for 14 days. For PD-1 Ab only treatment group and simultaneous combination treatment group, 0.2 mL of an administration sample containing 2.5 mg/mL of an anti-mouse-PD-1 antibody (Clone: RMP1-14, BioXCell, Catalog #: BE0146), which had been diluted with PBS, was intraperitoneally administered (at a dosage of 500 μg/head) once every 3 days a total of 5 times (day 1, day 4, day 7, day 10, and day 13, with the day of the grouping set to day 1). For lenvatinib then PD-1 Ab group, the same dose of an anti-mouse PD-1 antibody was intraperitoneally administrated once every 3 days a total of 3 times from day8 (day8, day11, day14).

To the control group, Otsuka water for injection was orally administered at a dose of 0.2 mL/20 g mouse body weight once daily for 14 days. Each group including 5 mice was used to conduct the experiment. The day (day 15) after the final administration, the respective tumor volumes TV were determined for the control group, the lenvatinib administration group, the anti-mouse-PD-1 antibody administration group, and the combination administration group. The values obtained by logarithmically transforming the tumor volumes were used to carry out statistical analysis.

Consistent with the above experiments, the combination of lenvatinib and anti-PD-1 demonstrates a synergistic effect between the two. Unexpectedly, the administration of lenvatinib only for 7 days followed by administration of lenvatinib and anti-PD-1 showed an even greater effect on reducing tumor volume than the combined administration of lenvatinib and anti-PD-1.

### Example 2: Anti-Tumor Effect by Co-Administration of Lenvatinib Mesilate and Anti-PD-L1 Antibody

A RPMI1640 culture medium containing 10% FBS and penicillin/streptomycin was used to culture a mouse colon carcinoma cell line CT26.WT (ATCC number: CRL-2638). Hank's Balanced Salt Solution (HBSS) was used to prepare a cell suspension having a concentration of 3.0×10⁷ cells/mL. The cell suspension was subcutaneously transplanted at a dose of 0.1 mL on the right lateral side of the body of each of 7-week-old mice (BALB/cAnNCrlCrlj, female, Charles River Laboratories Japan Inc.). 8 days after the transplantation, an electronic digital caliper (Digimatic (TM) Caliper; Mitutoyo Corporation) was used to measure the short and long diameters of a tumor of interest. The following equation was used to calculate the tumor volume TV.

$$Tumor Volume TV ( {mm}^{3} ) =$$

$$Long Diameter ( mm ) \times Short Diameter ( mm ) \times Short Diameter$$

$$( mm ) / 2.$$

Based on the tumor volumes on the first day of administration, grouping was carried out such that the average values of the tumor volumes were almost the same. A 1 mg/ml solution of lenvatinib mesilate was prepared using water for injection (Otsuka Pharmaceutical Co., Ltd.) and was orally administered at a dose of 0.2 mL/20 g mouse body weight once daily for 14 days. 0.2 mL of an administration sample containing 2.5 mg/mL of an anti-mouse-PD-L1 antibody, which had been diluted with PBS, was intraperitoneally administered (at a dosage of 500 μg/head) once every 3 days a total of 5 times (day 1, day 4, day 7, day 10, and day 13, with the day of the grouping set to day 1). To the control group, Otsuka water for injection was orally administered at a dose of 0.2 mL/20 g mouse body weight once daily for 14 days. Each group including 5 mice was used to conduct the experiment. The day (day 15) after the final administration, the respective tumor volumes TV were determined for the control group, the lenvatinib administration group, the anti-mouse-PD-L1 antibody administration group, and the combination administration group. The values obtained by logarithmically transforming the tumor volumes were used to carry out statistical analysis.

In the subcutaneous CT26.WT transplantation model, the combination of lenvatinib mesilate and the anti-mouse-PD-L1 antibody exhibited a significantly higher anti-tumor effect than either administered alone. For example, the combination group had a tumor volume that was at least two-fold less than the groups that received treatment with lenvatinib or anti-PD-L1. The daily change in the tumor volume is shown in Table 5. In addition, the tumor volumes the day after the final administration are shown in FIG. 8.

TABLE 5

| | Day 1 | Day 4 | Day 8 | Day 11 |
|---|---|---|---|---|
| Control group | 153 | 456 | 1320 | 1990 |
| Lenvatinib group | 154 | 472 | 1172 | 1555 |
| Anti-PD-L1 antibody group | 153 | 380 | 962 | 1557 |
| Combination group | 154 | 358 | 635 | 720 |

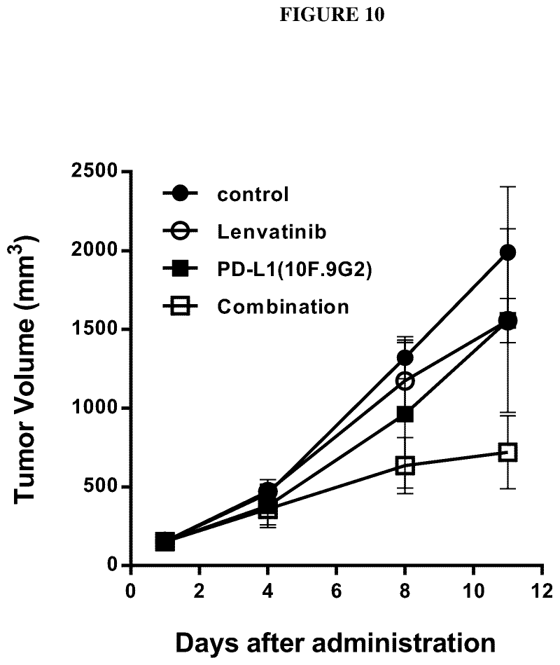

FIG. 10 shows a graph of the tumor volume plotted by days subsequent to administration for the control group, lenvatinib or PD-L1 individually, and a combination of lenvatinib and PD-L1. The combination of PD-L1 with lenvatinib exhibited a synergistic effect with respect to the tumor volume. The effect is noticeable at four days post commencement of treatment an very pronounced by days 8 and 11, with mice who received the combined lenvatinib and PD-L1 treatment showing a tumor volume nearly one third the size of the control group's tumor volume on day 11.

### REFERENCES

1. Sharpe, A. H, Wherry, E. J., Ahmed R., and Freeman G. J. The function of programmed cell death 1 and its ligands in regulating autoimmunity and infection. Nature Immunology (2007); 8:239-245.
2. Dong H et al. Tumor-associated B7-H1 promotes T-cell apoptosis: a potential mechanism of immune evasion. Nat Med. 2002 August; 8 (8): 793-800.
3. Yang et al. PD-1 interaction contributes to the functional suppression of T-cell responses to human uveal melanoma cells in vitro. Invest Ophthalmol Vis Sci. 2008 June; 49 (6 (2008): 49: 2518-2525.
4. Ghebeh et al. The B7-H1 (PD-L1) T lymphocyte-inhibitory molecule is expressed in breast cancer patients with infiltrating ductal carcinoma: correlation with important high-risk prognostic factors. Neoplasia (2006) 8:190-198.
5. Hamanishi J et al. Programmed cell death 1 ligand 1 and tumor-infiltrating CD8+T lymphocytes are prognostic factors of human ovarian cancer. Proceeding of the National Academy of Sciences (2007): 104: 3360-3365.
6. Thompson R H et al. Significance of B7-H1 overexpression in kidney cancer. Clinical genitourin Cancer (2006): 5: 206-211.
7. Nomi, T. Sho, M., Akahori, T., et al. Clinical significance and therapeutic potential of the programmed death-1 ligand/programmed death-1 pathway in human pancreatic cancer. Clinical Cancer Research (2007); 13:2151-2157.
8. Ohigashi Y et al. Clinical significance of programmed death-1 ligand-1 and programmed death-1 ligand 2 expression in human esophageal cancer. Clin. Cancer Research (2005): 11: 2947-2953.
9. Inman et al. PD-L1 (B7-H1) expression by urothelial carcinoma of the bladder and BCG-induced granulomata: associations with localized stage progression. Cancer (2007): 109: 1499-1505.
10. Shimauchi T et al. Augmented expression of programmed death-1 in both neoplasmatic and nonneoplastic CD4+ T-cells in adult T-cell Leukemia/Lymphoma. Int. J. Cancer (2007): 121:2585-2590.
11. Gao et al. Overexpression of PD-L1 significantly associates with tumor aggressiveness and postoperative recurrence in human hepatocellular carcinoma. Clinical Cancer Research (2009) 15:971-979.
12. Nakanishi J. Overexpression of B7-H1 (PD-L1) significantly associates with tumor grade and postoperative prognosis in human urothelial cancers. Cancer Immunol Immunother. (2007) 56:1173-1182.
13. Hino et al. Tumor cell expression of programmed cell death-1 is a prognostic factor for malignant melanoma. Cancer (2010): 00: 1-9.
14. Ghebeh H. Foxp3+tregs and B7-H1+/PD-1+T lymphocytes co-infiltrate the tumor tissues of high-risk breast cancer patients: implication for immunotherapy. BMC Cancer. 2008 Feb. 23; 8:57.
15. Ahmadzadeh M et al. Tumor antigen-specific CD8 T cells infiltrating the tumor express high levels of PD-1 and are functionally impaired. Blood (2009) 114:1537-1544.
16. Thompson R H et al. PD-1 is expressed by tumor infiltrating cells and is associated with poor outcome for patients with renal carcinoma. Clinical Cancer Research (2007) 15:1757-1761.
17. US Patent Application Publication No. 2004-053908.
18. US Patent Application Publication No. 2004-253205.
19. US Patent Application Publication No. 2010-105031.
20. US Patent Application Publication No. 2009-209580.
21. US Patent Application Publication No. 2009-264464.
22. US Patent Application Publication No. 2004-259834.
23. Iwai et al., PNAS, 2002, 99 (19), 12293-7.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57 (b) (1), to relate to each and every individual publication, database entry (e.g. Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1-18. (canceled)

19. A method for treating a human individual diagnosed with a cancer, comprising administering to the individual a combination therapy for at least 24 weeks, wherein the combination therapy pembrolizumab and lenvatinib or a pharmaceutically acceptable salt thereof, wherein lenvatinib or a pharmaceutically acceptable salt thereof is administered at a daily dose of 24 mg, 20 mg or 14 mg, each as lenvatinib, and pembrolizumab is administered at a dose of 200 mg once every three weeks.

20. The method of claim 19, wherein the individual is a human.

21. The method of claim 19, wherein the cancer is a solid tumor.

22. The method of claim 19, wherein the cancer is thyroid cancer, hepatocellular carcinoma (HCC), non-small cell lung cancer (NSCLC), renal cell carcinoma (RCC), endometrial cancer, urothelial cancer, squamous cell carcinoma of head and neck, glioblastoma, or melanoma.

23. The method of claim 19, wherein the pharmaceutically acceptable salt of lenvatinib is lenvatinib mesilate.

24. The method of claim 19, wherein the lenvatinib or a pharmaceutically acceptable salt thereof is administered at a daily dose of 24 mg, each as lenvatinib.

25. The method of claim 19, wherein the lenvatinib or a pharmaceutically acceptable salt thereof is administered at a daily dose of 20 mg, each as lenvatinib.

26. The method of claim 19, wherein the lenvatinib or a pharmaceutically acceptable salt thereof is administered at a daily dose of 14 mg, each as lenvatinib.

27. The method of claim 19 wherein the anti PD-1 antibody is pembrolizumab which is administered at a dose of 200 mg once every three weeks.

28. The method of claim 19, wherein the cancer is thyroid cancer.

29. The method of claim 19, wherein the cancer is HCC.

30. The method of claim 19, wherein the cancer is RCC.

31. The method of claim 19, wherein the cancer is endometrial cancer.

32. The method of claim 19, wherein the cancer is squamous cell carcinoma of head and neck.

33. The method of claim 19, wherein the cancer is glioblastoma.

34. The method of claim 19, wherein the cancer is gastric cancer.

35. The method of claim 19, wherein the cancer is melanoma.


SEQUENCE LISTING (25 sequences)

SEQ ID NO: 1 - protein, 15 aa, synthetic construct
  REGION 1..15: note = Antibody Light Chain CDR
RASKSVSTSGFSYLH

SEQ ID NO: 2 - protein, 7 aa, synthetic construct
  REGION 1..7: note = Antibody Light Chain CDR
LASNLES

SEQ ID NO: 3 - protein, 9 aa, synthetic construct
  REGION 1..9: note = Antibody Light Chain CDR
QHSWELPLT

SEQ ID NO: 4 - protein, 5 aa, synthetic construct
  REGION 1..5: note = Antibody Heavy Chain CDR
SYYLY

SEQ ID NO: 5 - protein, 17 aa, synthetic construct
  REGION 1..17: note = Antibody Heavy Chain CDR
GVNPSNGGTNFSEKFKS

SEQ ID NO: 6 - protein, 11 aa, synthetic construct
  REGION 1..11: note = Antibody Heavy Chain CDR
RDSNYDGGFDY

SEQ ID NO: 7 - protein, 15 aa, synthetic construct
  REGION 1..15: note = Antibody Light Chain CDR
RASKGVSTSGYSYLH

SEQ ID NO: 8 - protein, 7 aa, synthetic construct
  REGION 1..7: note = Antibody Light Chain CDR
LASYLES

SEQ ID NO: 9 - protein, 9 aa, synthetic construct
  REGION 1..9: note = Antibody Light Chain CDR
QHSRDLPLT

SEQ ID NO: 10 - protein, 5 aa, synthetic construct
  REGION 1..5: note = Antibody Heavy Chain CDR
NYYMY

SEQ ID NO: 11 - protein, 17 aa, synthetic construct
  REGION 1..17: note = Antibody Heavy Chain CDR
GINPSNGGTNFNEKFKN

SEQ ID NO: 12 - protein, 11 aa, synthetic construct
  REGION 1..11: note = Antibody Heavy Chain CDR
RDYRFDMGFDY

SEQ ID NO: 13 - protein, 120 aa, synthetic construct [sequence omitted]

SEQ ID NO: 14 - protein, 447 aa, synthetic construct
  REGION 1..447: note = Humanized Antibody Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 111 aa, synthetic construct [sequence omitted]

SEQ ID NO: 16 - protein, 111 aa, synthetic construct
  REGION 1..111: note = Humanized Antibody Light Chain Variable Region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 17 - protein, 111 aa, synthetic construct
  REGION 1..111: note = Humanized Antibody Light Chain Variable Regon
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 18 - protein, 218 aa, synthetic construct
  REGION 1..218: note = Humanzed Antiboy LIght Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 218 aa, synthetic construct
  REGION 1..218: note = Humanized Antibody Light Chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 218 aa, synthetic construct
  REGION 1..218: note = Humanized Antibody Light Chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct; identical to SEQ ID NO: 14
  REGION 1..447: note = Humanized Antibody Heavy Chain

SEQ ID NO: 22 - protein, 218 aa, synthetic construct; identical to SEQ ID NO: 18
  REGION 1..218: note = Humanized Antibody Light Chain

SEQ ID NO: 23 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 290 aa, Homo sapiens
  REGION 19..290: note = Mature Peptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET